(19) United States
(12) Patent Application Publication
(10) Pub. No.: US 2025/0289814 A1
(43) Pub. Date: Sep. 18, 2025

(54) COMPOUNDS AS PARP1 INHIBITIORS

(71) Applicant: NINGBO NEWBAY TECHNOLOGY DEVELOPMENT CO., LTD., Zhejiang (CN)

(72) Inventors: Tingzhong WANG, Zhejiang (CN); Zhenhai SHEN, Zhejiang (CN); Eric P A TALBOT, Hertfordshire (GB); Joseph M. BATEMAN, Hertfordshire (GB); Benjamin F. RAHEMTULLA, Hertfordshire (GB); Mustafa MOROGLU, Hertfordshire (GB); Tammy LADDUWAHETTY, Hertfordshire (GB)

(21) Appl. No.: 18/861,016

(22) PCT Filed: Feb. 20, 2023
(86) PCT No.: PCT/CN2023/077272

§ 371 Date: Oct. 28, 2024

(30) Foreign Application Priority Data
Apr. 28, 2022 (WO) PCT/CN2022/089986

(51) Int. Cl. C07D 471/04; A61K 31/444; A61K 31/498; A61P 35/00; C07D 401/14

(52) U.S. Cl. CPC C07D 471/04; A61K 31/444; A61K 31/498; A61P 35/00; C07D 401/14

(57) ABSTRACT

Disclosed herein is the compounds and pharmaceutically acceptable salts thereof that inhibit the Poly (ADP-ribose) polymerase (PARP) family of enzymes. The present disclosure also relates to the use of these compounds or pharmaceutically acceptable salts thereof in the treatment of diseases.

### TECHNICAL FIELD

The present invention is related to compounds and pharmaceutically acceptable salts thereof that inhibit the Poly (ADP-ribose) polymerase (PARP) family of enzymes. The present disclosure also relates to the use of these compounds or pharmaceutically acceptable salts thereof in the treatment of diseases.

### BACKGROUND OF THE INVENTION

The family of poly (ADP-ribose) polymerases (PARP) is composed of about 17 proteins, including PARP-1, PARP-2, PARP-3, PARP-4 (vPARP), PARP-5 (tankyrase-1, tankyrase-2), PARP-7, PARP-10 and the like. These proteins all show a certain level of homology in their catalytic domain but differ in their cellular functions.

Among the many functions attributed to PARP-1 and PARP-2, its major role is to facilitate DNA repair by ADP-ribosylation and therefore coordinate a number of DNA repair proteins. Activation of PARP is induced by DNA single strand breaks after exposure to radiation, oxygen free radicals, or nitric oxide (NO), etc. DNA damage leads to PARP activation that repairs DNA single strand breaks, and thus PARP can contribute to resistance that may occur in various types in cancer therapy. Particularly, PARP inhibitors were reported to be useful for specific killing of tumors deficient in DNA double-strand repair factors such as BRCA-1 and BRCA-2, and thus have been developed as patient-specific anticancer agents against various types of cancers, including breast cancer, ovarian cancer, prostate cancer and the like, which have abnormalities in DNA double-strand damage repair factors.

Inhibition of PARP family enzymes has been exploited as a strategy to selectively kill cancer cells by inactivating complementary DNA repair pathways. A number of pre-clinical and clinical studies have demonstrated that tumour cells bearing deleterious alterations of BRCA1 or BRCA2, key tumour suppressor proteins involved in double-strand DNA break (DSB) repair by homologous recombination (HR), are selectively sensitive to small molecule inhibitors of the PARP family of DNA repair enzymes. Such tumours have deficient homologous recombination repair (HRR) pathways and are dependent on PARP enzymes function for survival. Although PARP inhibitor therapy has predominantly targeted BRCA-mutated cancers, PARP inhibitors have been tested clinically in non-BRCA-mutant tumors, those which exhibit homologous recombination deficiency (HRD).

It is believed that PARP inhibitors having improved selectivity for PARP1 may possess improved efficacy and reduced toxicity compared to other clinical PARP1/2 inhibitors. It is believed also that selective strong inhibition of PARP1 would lead to trapping of PARP1 on DNA, resulting in DNA double strand breaks (DSBs) through collapse of replication forks in S-phase. It is believed also that PARP1—DNA trapping is an effective mechanism for selectively killing tumour cells having HRD. An unmet medical need therefore exists for effective and safe PARP inhibitors. Especially PARP inhibitors having selectivity for PARP1.

### SUMMARY OF THE INVENTION

The present invention is related to compounds or pharmaceutically acceptable salts that have PARP inhibitory activity, and therefore may be useful for the treatment of diseases and conditions in which PARP function has pharmacological significance. Furthermore, compounds described herein have high selectivity for PARP1 over PARP2.

In one aspect, the present invention provides compounds or pharmaceutically acceptable salts that have PARP inhibitory activity. Preferably, the compounds described herein have high selectivity for PARP1 over other PARP family members such as PARP2.

In another aspect, the present invention provides a composition comprising compounds of formula I or a pharmaceutically acceptable salt. Preferably, the composition further comprising at least one pharmaceutically acceptable diluent, excipient or inert carrier.

In another aspect, the present invention provides compounds or pharmaceutically acceptable salts thereof, or a composition thereof, for use as a medicament.

In another aspect, the present invention provides a method of treatment comprising administration of a therapeutically effective amount of compound thereof to a patient in need. Preferably, the patient in need has cancer. Preferably, the cancer is deficient in HR dependent DNA DSB repair pathway. More preferably, the cancer cells have a BRCA1 or BRCA2 deficient phenotype, or the cancer cells are deficient in BRCA1 or BRCA2. More preferably, the cancer is selected from anyone of breast, ovary, pancreas, prostate, hematological, gastrointestinal, and lung cancer.

In another aspect, the present invention provides a method of treatment or prophylaxis of diseases and conditions in which inhibition of PARP1 is beneficial comprising administration of a therapeutically effective amount of compound thereof to a patient in need. Preferably, the patient in need has cancer. More preferably, the patient is heterozygous for a mutation in a gene encoding a component of the HR dependent DNA DSB repair pathway, or the patient is heterozygous for a mutation in a gene encoding a component of the HR dependent DNA DSB repair pathway.

In another aspect, the present invention provides use of the compound in the manufacture of a medicament for use in the treatment of cancer. More preferably, the cancer is deficient in HR dependent DNA DSB repair pathway.

### DETAILED DESCRIPTION OF THE INVENTION

### (I) Compound

In one aspect, the present invention provides compounds of formula I or pharmaceutically acceptable salts thereof that have PARP inhibitory activity. Preferably, the compounds described herein have high selectivity for PARP1 over other PARP family members such as PARP2.

In some embodiments, the compound is a compound of formula I:

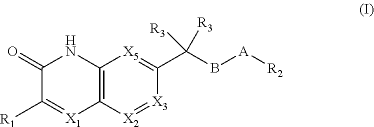

or a pharmaceutically acceptable salt thereof, wherein
X₁, X₂ or X₃ is independently selected from the group consisting of N or CH;
X₅ is selected from the group consisting of N, CH or CF;
A is selected from the group consisting of —CH₂—, —O— or —NR₇—, wherein R₇ is selected from the group consisting of H or C₁-C₆ alkyl, or A is absent;
B is selected from the group consisting of one substituted or unsubstituted

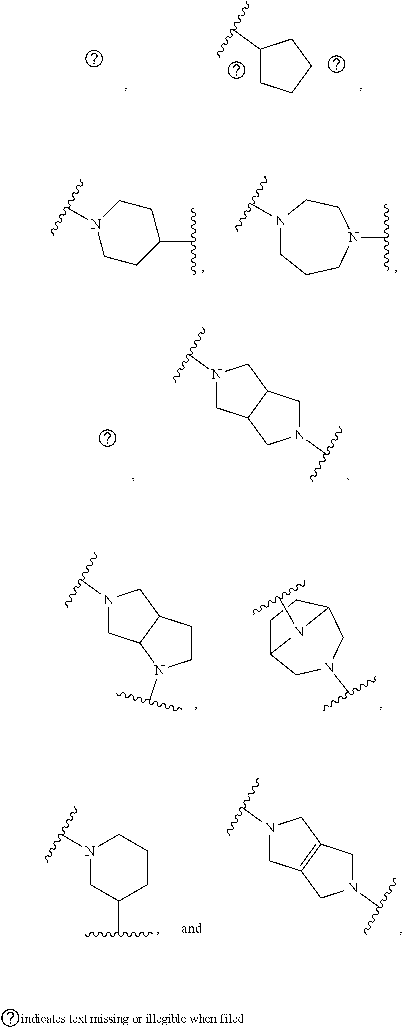

the substituted group at any position of ring B is R₅;
R₁ is selected from the group consisting of C₁-C₆ alkyl, C₁-C₆ haloalkyl, C₁-C₆ alkoxy, C₃-C₅ cycloalkyl;
R₂ is

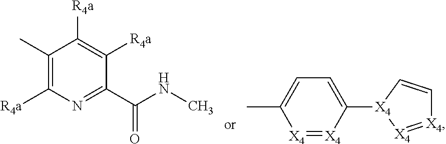

wherein one X₄ is N and one X₄ is CH;
each R₃ is independently selected from the group consisting of H or unsubstituted or substituted —C₁-C₆ alkyl, the substitutes of —C₁-C₆ alkyl is selected from the group consisting of H, —O—CH₃, —CN, —OH, or two R₃ are attached to form a C₃-C₅ cycloalkyl;
each R₄ₐ is independently selected from the group consisting of H, CN, halogen, C₁-C₆ alkyl, —O-alkyl, C₁-C₆ haloalkyl, or —C₁-C₆ alkoxy; preferably, R₄ₐ is independently selected from the group consisting of H, CN, halogen, C₁-C₃ alkyl, —O—C₁-C₃alkyl, C₁-C₃ haloalkyl, or —C₁-C₃ alkoxy; more preferably, each R₄ₐ is independently selected from the group consisting of H, —CH₃, —CN, F.

R₅ is selected from the group consisting of H, C₁-C₆ alkyl, ═O, —(CH₂)₁₋₃OH, or halogen.

In some embodiments, the compound of formula I is selected from the group consisting of

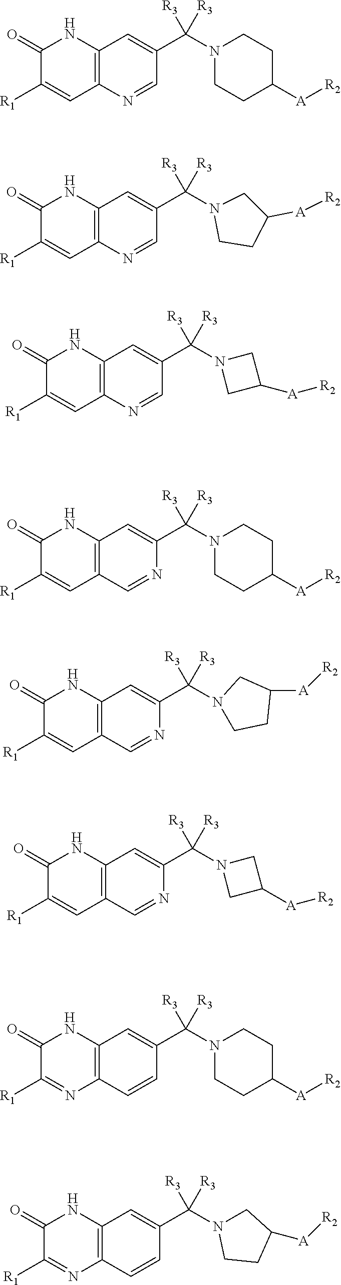

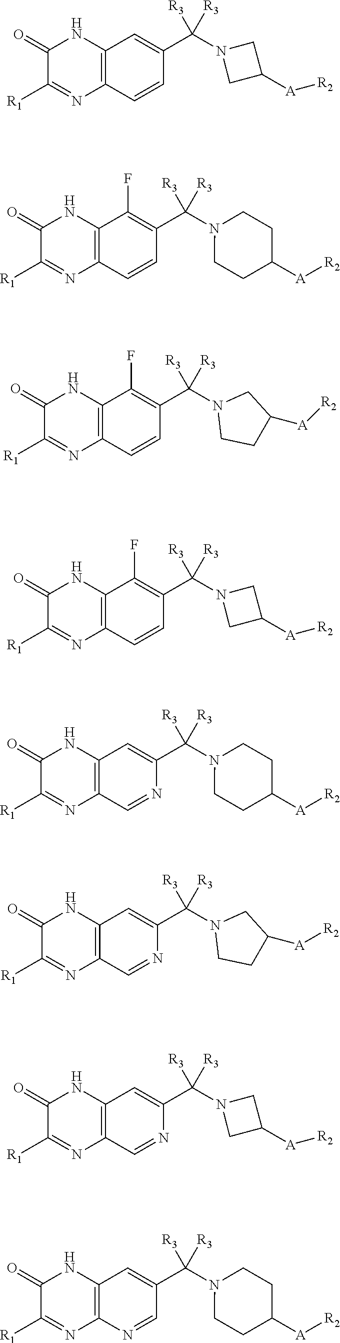

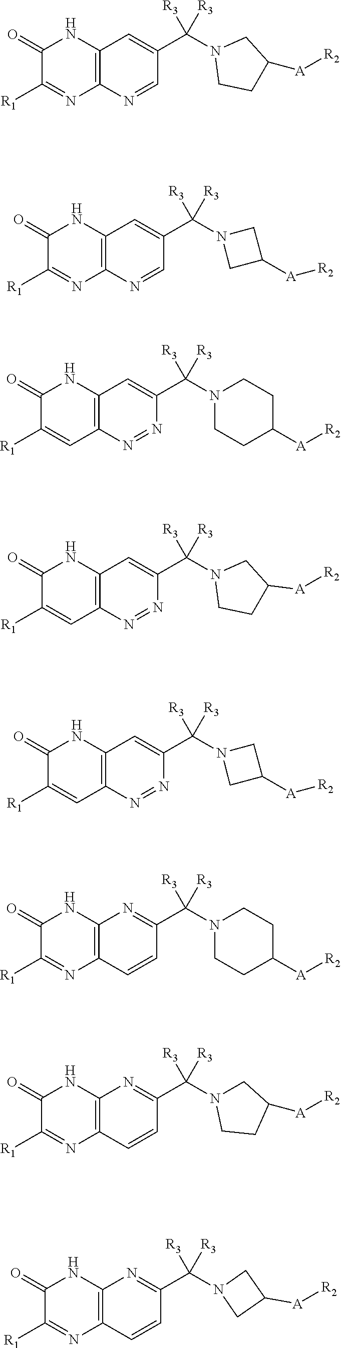

or a pharmaceutically acceptable salt thereof.

In some embodiments, the compound of formula I, wherein,
A is —O—;
B is selected from the group consisting of one substituted or unsubstituted

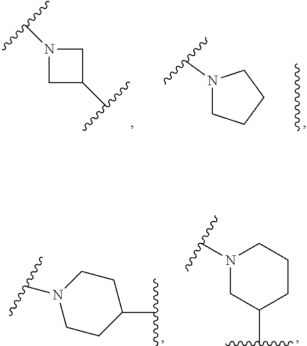

and the substituted group at any position of ring B is R₅;
R₁ is selected from the group consisting of C₁-C₃ alkyl, C₁-C₃ haloalkyl, C₁-C₃ alkoxy, C₃-C₅ cycloalkyl;
R₂ is

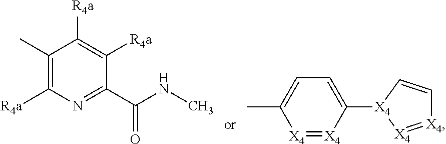

wherein one X₄ is N and one X₄ is CH;
each R₃ is independently selected from the group consisting of H or unsubstituted or substituted —C₁-C₆ alkyl, the substitutes of —C₁-C₆ alkyl is selected from the group consisting of H, —O—CH₃, —CN, —OH, or two R₃ are attached to form a C₃-C₆ cycloalkyl;
each R₄ₐ is independently selected from the group consisting of H, CN, halogen, C₁-C₃ alkyl, —O—C₁-C₃alkyl, C₁-C₃ haloalkyl, or —C₁-C₃ alkoxy; preferably, each R₄ₐ is independently selected from the group consisting of H, —CH₃, —CN, F;
R₅ is selected from the group consisting of —CH₃, —CH₂OH, or —F.

In some embodiments, the compound of formula I, wherein the formula I is

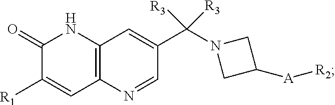

A is selected from —O—;
R₁ is selected from the group consisting of C₁-C₃ alkyl, C₃-C₅ cycloalkyl;
R₂ is

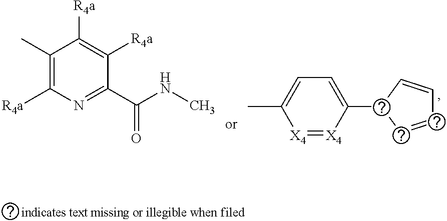

wherein one X₄ is N and one X₄ is CH;
each R₃ is independently selected from the group consisting of H or unsubstituted or substituted —C₁-C₆ alkyl, the substitutes of —C₁-C₆ alkyl is selected from the group consisting of H, —O—CH₃, —CN, —OH, or two R₃ are attached to form a C₃-C₅ cycloalkyl;
each R₄ₐ is independently selected from the group consisting of H, —CH₃, —CN, F.

In some embodiments, the compound of formula I, wherein two R₃ are not H simultaneously.

In some embodiments, the compound of formula I, wherein

R₂ is

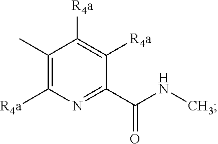

each R₄ₐ is independently selected from the group consisting of H, —CH₃, —CN, F;
each R₃ is independently selected from the group consisting of H or —C₁-C₃ alkyl, wherein two R₃ are not H simultaneously.

In some embodiments, the compound of formula I, wherein the formula I is

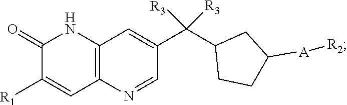

A is selected from —O—;
R₁ is selected from the group consisting of C₁-C₃ alkyl, C₃-C₅ cycloalkyl;
R₂ is

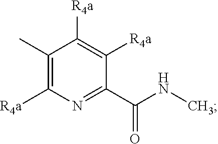

each R₃ is independently selected from the group consisting of H or unsubstituted or substituted —C₁-C₆ alkyl, the substitutes of —C₁-C₆ alkyl is selected from the group consisting of H, —O—CH₃, —CN, —OH, or two R₃ are attached to form a C₃-C₅ cycloalkyl;
each R₄ₐ is independently selected from the group consisting of H, —CH₃, —CN, F.

In some embodiments, the compound of formula I wherein, preferably,
R₁ is selected from the group consisting of C₁-C₃ alkyl;
each R₃ is independently selected from the group consisting of H or unsubstituted —C₁-C₃ alkyl;
each R₄ₐ is independently selected from the group consisting of H, —CH₃, F.

In some embodiments, the compound of formula I is selected from table 1, or a pharmaceutically acceptable salt thereof.

TABLE 1

| Compound | | | LC-MS (ES+) |
|---|---|---|---|
| No. | Structure | Name | m/z |
| 1 | 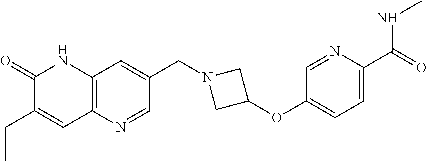 [SMILES: CCc1cc2ncc(CN3CC(Oc4ccc(C(=O)NC)nc4)C3)cc2[nH]c1=O] | 5-((1-((7-ethyl-6-oxo-5,6- dihydro-1,5-naphthyridin- 3-yl)methyl) azetidin-3-yl)oxy)-N- methylpicolinamide | 394.3 |
| 2 | 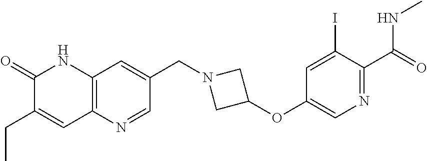 [SMILES: CCc1cc2ncc(CN3CC(Oc4cnc(C(=O)NC)c(I)c4)C3)cc2[nH]c1=O] | 5-((1-((7-ethyl-6-oxo- 5,6-dihydro-1,5- naphthyridin-3-yl)methyl) azetidin-3-yl)oxy)-3-fluoro- N-methylpicolinamide | 412.35 |
| 3 | 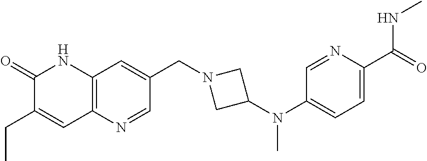 [SMILES: CCc1cc2ncc(CN3CC(N(C)c4ccc(C(=O)NC)nc4)C3)cc2[nH]c1=O] | 5-((1-((7-ethyl-6-oxo-5, 6-dihydro-1,5-naphthyridin- 3-yl)methyl)azetidin- 3-yl)(methyl)amino)- N-methylpicolinamide | 407.2 |
| 4 | 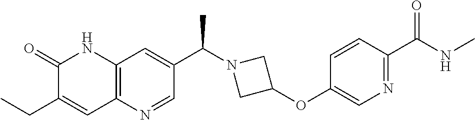 [SMILES: CCc1cc2ncc([C@@H](C)N3CC(Oc4ccc(C(=O)NC)nc4)C3)cc2[nH]c1=O] | (R)-5-((1-(1-(7-ethyl-6-oxo- 5,6-dihydro-1,5-naphthyridin- 3-yl)ethyl)azetidin-3- yl)oxy)-N- methylpicolinamide | 408.35 |
| 5 | 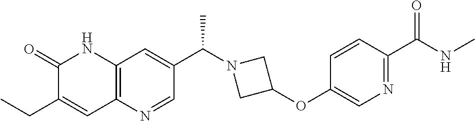 [SMILES: CCc1cc2ncc([C@H](C)N3CC(Oc4ccc(C(=O)NC)nc4)C3)cc2[nH]c1=O] | (S)-5-((1-(1-(7-ethyl-6- oxo-5,6-dihydro-1,5- naphthyridin-3-yl)ethyl) azetidin-3-yl)oxy)-N- methylpicolinamide | 408.35 |
| 6 | 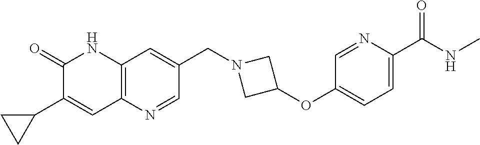 [SMILES: CNC(=O)c1ccc(OC2CN(Cc3cnc4cc(C5CC5)c(=O)[nH]c4c3)C2)cn1] | 5-((1-((7-cyclopropyl-6- oxo-5,6-dihydro-1,5- naphthyridin-3-yl)methyl) azetidin-3-yl)oxy)-N- methylpicolinamide | 406.2 |
| 7 | 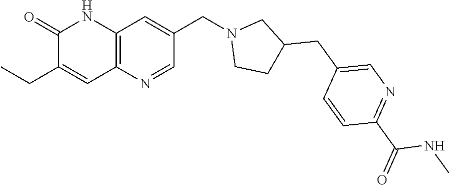 [SMILES: CCc1cc2ncc(CN3CCC(Cc4ccc(C(=O)NC)nc4)C3)cc2[nH]c1=O] | 5-((1-((7-ethyl-6-oxo-5, 6-dihydro-1,5-naphthyridin- 3-yl)methyl)pyrrolidin- 3-yl)methyl)-N- methylpicolinamide | 406.4 |
| 8 | 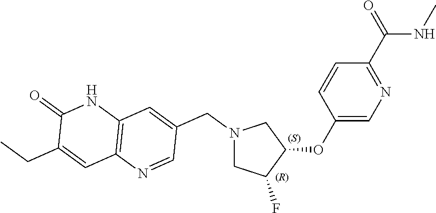 | 5-(((3S, 4R)-1-((7-ethyl-6-oxo- 5,6-dihydro-1,5- naphthyridin-3-yl)methyl)- 4-fluoropyrrolidin-3-yl)oxy)- N-methylpicolinamide | 426.3 |
| 9 | 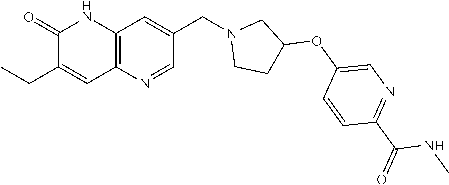 [SMILES: CCc1cc2ncc(CN3CCC(Oc4ccc(C(=O)NC)nc4)C3)cc2[nH]c1=O] | 5-((1-((7-ethyl-6-oxo-5,6- dihydro-1,5-naphthyridin- 3-yl)methyl)pyrrolidin- 3-yl)oxy)-N- methylpicolinamide | 408.2 |
| 10 | 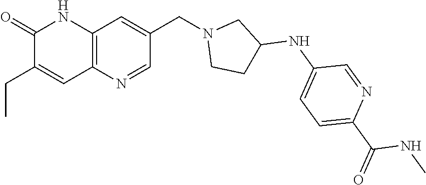 [SMILES: CCc1cc2ncc(CN3CCC(Nc4ccc(C(=O)NC)nc4)C3)cc2[nH]c1=O] | 5-((1-((7-ethyl-6-oxo-5,6- dihydro-1,5-naphthyridin- 3-yl)methyl)pyrrolidin- 3-yl)amino)-N- methylpicolinamide | 407.3 |
| 11 | 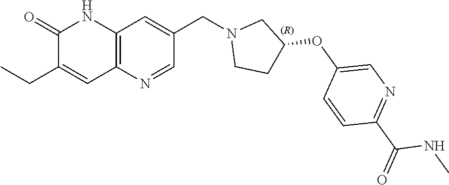 | (R)-5-((1-((7-ethyl-6- oxo-5,6-dihydro-1,5- naphthyridin-3-yl)methyl) pyrrolidin-3-yl)oxy)-N- methylpicolinamide | 408.2 |
| 12 and 13 | 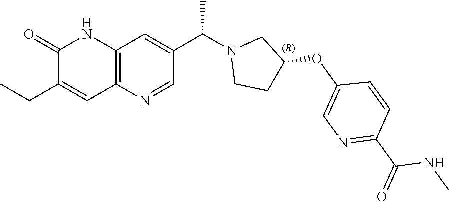 | 5-(((R)-1-((enantiomer 1 or 2)-1-(7-ethyl-6-oxo-5,6- dihydro-1,5-naphthyridin- 3-yl)ethyl)pyrrolidin- 3-yl)oxy)-N- methylpicolinamide | 422.2 422.2 |
| | 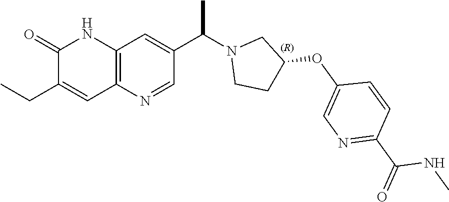 | | |
| 14 | 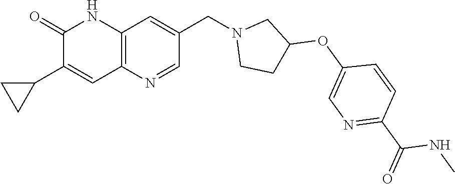 [SMILES: CNC(=O)c1ccc(OC2CCN(Cc3cnc4cc(C5CC5)c(=O)[nH]c4c3)C2)cn1] | 5-((1-((7-cyclopropyl-6- oxo-5,6-dihydro-1,5- naphthyridin-3-yl)methyl) pyrrolidin-3-yl)oxy)- N-methylpicolinamide | 420.1 |
| 15 | 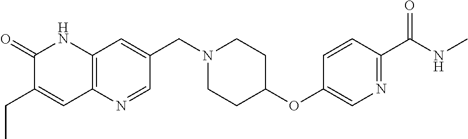 [SMILES: CCc1cc2ncc(CN3CCC(Oc4ccc(C(=O)NC)nc4)CC3)cc2[nH]c1=O] | 5-((1-((7-ethyl-6-oxo-5,6- dihydro-1,5-naphthyridin- 3-yl)methyl)piperidin- 4-yl)oxy)-N- methylpicolinamide | 422.1 |
| 16 and 17 | 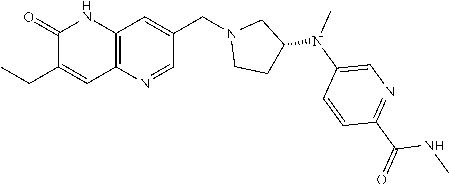 [SMILES: CCc1cc2ncc(CN3CC[C@@H](N(C)c4ccc(C(=O)NC)nc4)C3)cc2[nH]c1=O] | (enantiomer 1 or 2)-5-((1-((7-ethyl-6-oxo- 5,6-dihydro-1,5- naphthyridin-3-yl)methyl) pyrrolidin-3-yl)(methyl) amino)-N- methylpicolinamide | 421.3 421.3 |
| | 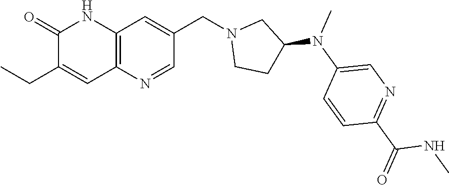 [SMILES: CCc1cc2ncc(CN3CC[C@H](N(C)c4ccc(C(=O)NC)nc4)C3)cc2[nH]c1=O] | | |
| 18 | 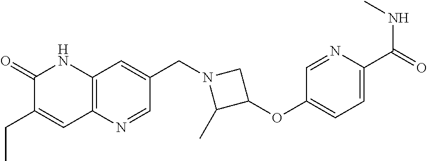 [SMILES: CCc1cc2ncc(CN3CC(Oc4ccc(C(=O)NC)nc4)C3C)cc2[nH]c1=O] | 5-((1-((7-ethyl-6-oxo-5,6- dihydro-1,5-naphthyridin-3- yl)methyl)-2-methylazetidin- 3-yl)oxy)-N- methylpicolinamide | 408.2 |
| 19 | 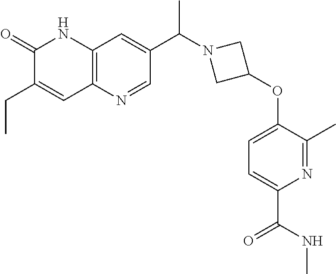 [SMILES: CCc1cc2ncc(C(C)N3CC(Oc4ccc(C(=O)NC)nc4C)C3)cc2[nH]c1=O] | 5-((1-(1-(7-ethyl-6-oxo-5,6- dihydro-1,5-naphthyridin- 3-yl)ethyl)azetidin-3- yl)oxy)-N,6- dimethylpicolinamide | 422.1 |
| 20 | 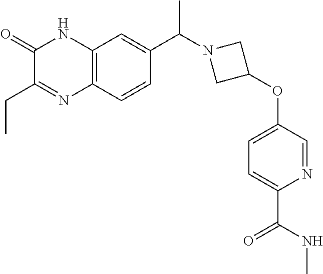 [SMILES: CCc1nc2ccc(C(C)N3CC(Oc4ccc(C(=O)NC)nc4)C3)cc2[nH]c1=O] | 5-((1-(1-(2-ethyl-3-oxo-3,4- dihydroquinoxalin-6-yl) ethyl)azetidin-3-yl) oxy)-N-methylpicolinamide | 408.2 |
| 21 | 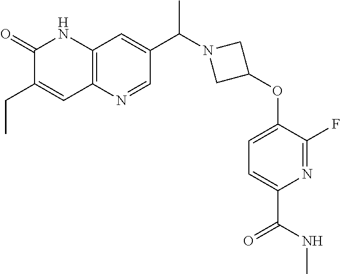 [SMILES: CCc1cc2ncc(C(C)N3CC(Oc4ccc(C(=O)NC)nc4F)C3)cc2[nH]c1=O] | 5-((1-(1-(7-ethyl-6-oxo-5,6- dihydro-1,5-naphthyridin- 3-yl)ethyl)azetidin-3- yl)oxy)-6-fluoro-N- methylpicolinamide | 426.2 |
| 22 and 23 | 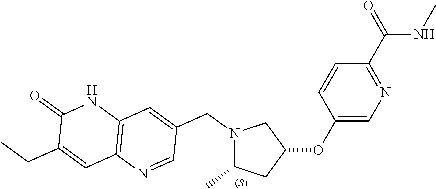 | 5-(((enantiomer 1 or 2, 5S)-1-((7-ethyl-6-oxo-5, 6-dihydro-1,5-naphthyridin- 3-yl)methyl)-5-methyl pyrrolidin-3-yl)oxy)-N- methylpicolinamide | 422.3 422.3 |
| | 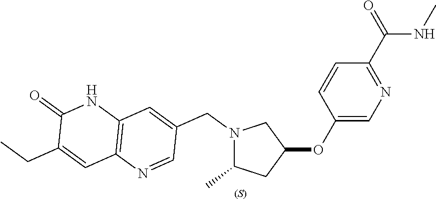 | | |
| 24 | 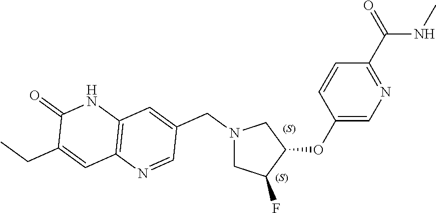 | 5-(((3S,4S)-1-((7-ethyl-6- oxo-5,6-dihydro-1,5- naphthyridin-3-yl)methyl)- 4-fluoropyrrolidin-3-yl) oxy)-N-methylpicolinamide | 426.4 |
| 25 and 26 | 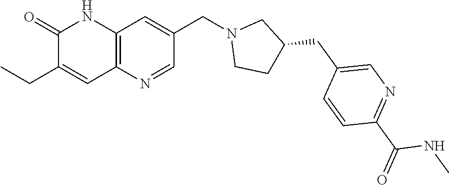 [SMILES: CCc1cc2ncc(CN3CC[C@@H](Cc4ccc(C(=O)NC)nc4)C3)cc2[nH]c1=O] | (enantiomer 1 or 2)-5-((1-((7-ethyl-6-oxo- 5,6-dihydro-1,5-naphthyridin- 3-yl)methyl)pyrrolidin- 3-yl)methyl)-N- methylpicolinamide | 406.4 406.4 |
| | 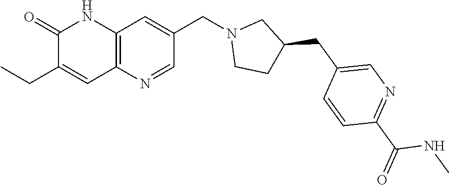 [SMILES: CCc1cc2ncc(CN3CC[C@H](Cc4ccc(C(=O)NC)nc4)C3)cc2[nH]c1=O] | | |
| 27 | 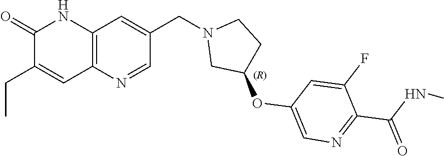 | (R)-5-((1-((7-ethyl-6-oxo- 5,6-dihydro-1,5-naphthyridin- 3-yl)methyl)pyrrolidin- 3-yl)oxy)-3-fluoro-N- methylpicolinamide | 426.1 |
| 28 | 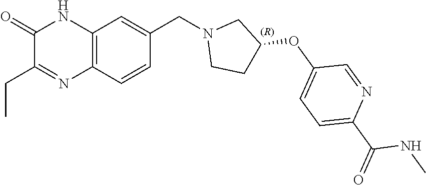 | (R)-5-((1-((2-ethyl-3-oxo- 3,4-dihydroquinoxalin-6- yl)methyl)pyrrolidin-3- yl)oxy)-N- methylpicolinamide | 408.3 |
| 29 | 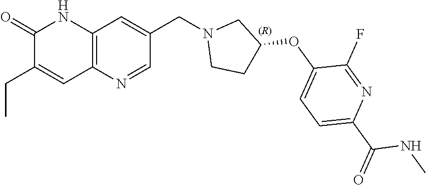 | (R)-5-((1-((7-ethyl-6-oxo- 5,6-dihydro-1,5-naphthyridin- 3-yl)methyl)pyrrolidin- 3-yl)oxy)-6-fluoro-N- methylpicolinamide | 426.1 |
| 30 | 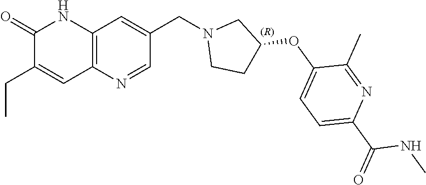 | (R)-5-((1-((7-ethyl-6-oxo- 5,6-dihydro- 1,5-naphthyridin- 3-yl)methyl)pyrrolidin- 3-yl)oxy)-N,6- dimethylpicolinamide | 422.3 |
| 31 | 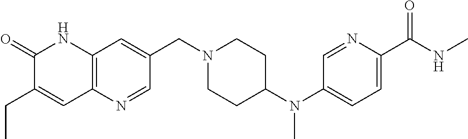 [SMILES: CCc1cc2ncc(CN3CCC(N(C)c4ccc(C(=O)NC)nc4)CC3)cc2[nH]c1=O] | 5-((1-((7-ethyl-6-oxo-5,6- dihydro-1,5-naphthyridin- 3-yl)methyl)piperidin- 4-yl)(methyl)amino)-N- methylpicolinamide | 435.3 |
| 32 and 33 | 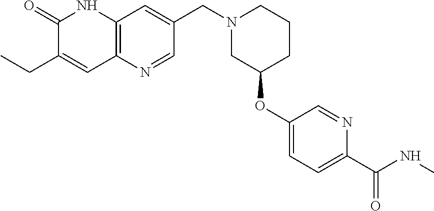 [SMILES: CCc1cc2ncc(CN3CCC[C@@H](Oc4ccc(C(=O)NC)nc4)C3)cc2[nH]c1=O] | (enantiomer 1 or 2)-5-((1-((7-ethyl-6-oxo- 5,6-dihydro-1,5-naphthyridin- 3-yl)methyl)piperidin- 3-yl)oxy)-N- methylpicolinamide | 422.3 422.3 |
| | 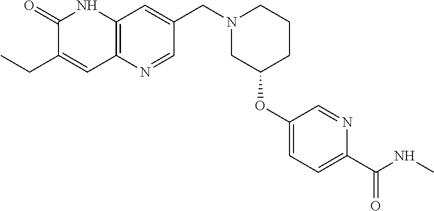 [SMILES: CCc1cc2ncc(CN3CCC[C@H](Oc4ccc(C(=O)NC)nc4)C3)cc2[nH]c1=O] | | |
| 34 | 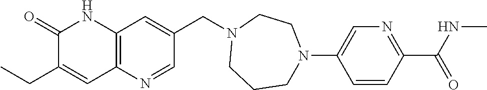 [SMILES: CCc1cc2ncc(CN3CCCN(c4ccc(C(=O)NC)nc4)CC3)cc2[nH]c1=O] | 5-(4-((7-ethyl-6-oxo-5,6- dihydro-1,5-naphthyridin- 3-yl)methyl)-1,4-diazepan- 1-yl)-N-methylpicolinamide | 421.2 |
| 35 | 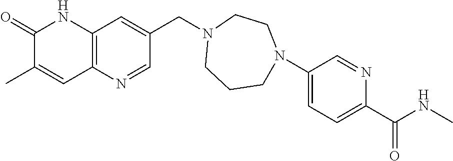 [SMILES: CNC(=O)c1ccc(N2CCCN(Cc3cnc4cc(C)c(=O)[nH]c4c3)CC2)cn1] | N-methyl-5-(4-((7-methyl- 6-oxo-5,6-dihydro-1,5- naphthyridin-3-yl)methyl)- 1,4-diazepan-1-yl) picolinamide | 407.1 |
| 36 | 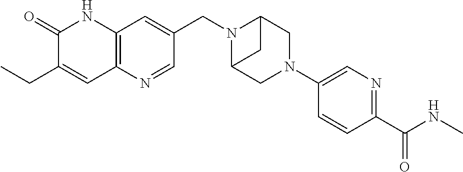 [SMILES: CCc1cc2ncc(CN3C4CC3CN(c3ccc(C(=O)NC)nc3)C4)cc2[nH]c1=O] | 5-(6-((7-ethyl-6-oxo-5,6- dihydro-1,5-naphthyridin- 3-yl)methyl)-3,6-diazabicyclo [3.1.1]heptan-3-yl)- N-methylpicolinamide | 419.2 |
| 37 | 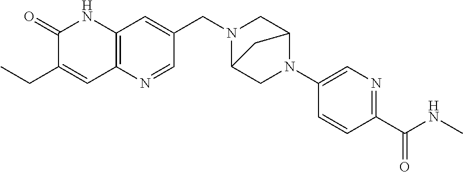 [SMILES: CCc1cc2ncc(CN3CC4CC3CN4c3ccc(C(=O)NC)nc3)cc2[nH]c1=O] | 5-(5-((7-ethyl-6-oxo-5,6- dihydro-1,5-naphthyridin- 3-yl)methyl)-2,5-diazabicyclo [2.2.1]heptan-2-yl)- N-methylpicolinamide | 419.3 |
| 38 | 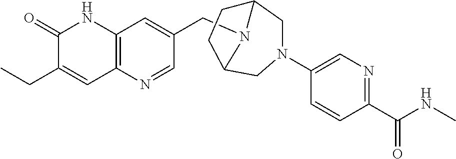 [SMILES: CCc1cc2ncc(CN3C4CCC3CN(c3ccc(C(=O)NC)nc3)C4)cc2[nH]c1=O] | 5-(8-((7-ethyl-6-oxo-5,6- dihydro-1,5-naphthyridin- 3-yl)methyl)-3,8-diazabicyclo [3.2.1]octan-3-yl)- N-methylpicolinamide | 433.2 |
| 39 | 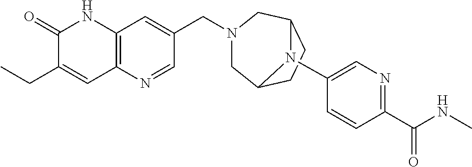 [SMILES: CCc1cc2ncc(CN3CC4CCC(C3)N4c3ccc(C(=O)NC)nc3)cc2[nH]c1=O] | 5-(3-((7-ethyl-6-oxo-5,6- dihydro-1,5-naphthyridin- 3-yl)methyl)-3,8-diazabicyclo [3.2.1]octan-8-yl)- N-methylpicolinamide | 433.3 |
| 40 | 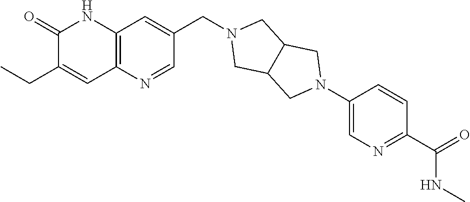 [SMILES: CCc1cc2ncc(CN3CC4CN(c5ccc(C(=O)NC)nc5)CC4C3)cc2[nH]c1=O] | 5-(5-((7-ethyl-6-oxo-5,6- dihydro-1,5-naphthyridin- 3-yl)methyl) hexahydropyrrolo [3,4-c]pyrrol-2(1H)- yl)-N-methylpicolinamide | 433.3 |
| 41 | 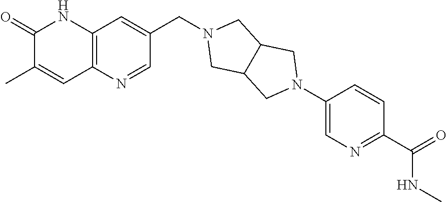 [SMILES: CNC(=O)c1ccc(N2CC3CN(Cc4cnc5cc(C)c(=O)[nH]c5c4)CC3C2)cn1] | N-methyl-5-(5-((7-methyl- 6-oxo-5,6-dihydro-1,5- naphthyridin-3-yl)methyl) hexahydropyrrolo[3,4-c] pyrrol-2(1H)-yl)picolinamide | 419.1 |
| 42 | 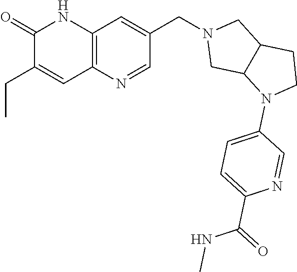 [SMILES: CCc1cc2ncc(CN3CC4CCN(c5ccc(C(=O)NC)nc5)C4C3)cc2[nH]c1=O] | 5-(5-((7-ethyl-6-oxo-5,6- dihydro-1,5-naphthyridin- 3-yl)methyl) hexahydropyrrolo[3,4- b]pyrrol-1(2H)- yl)-N-methylpicolinamide | 433.2 |
| 43 | 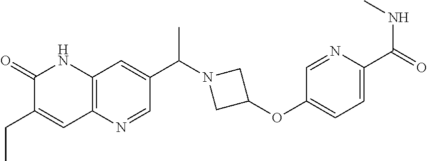 [SMILES: CCc1cc2ncc(C(C)N3CC(Oc4ccc(C(=O)NC)nc4)C3)cc2[nH]c1=O] | 5-((1-(1-(7-ethyl-6-oxo-5,6- dihydro-1,5-naphthyridin- 3-yl)ethyl)azetidin-3- yl)oxy)-N- methylpicolinamide | 408.4 |
| 44 | 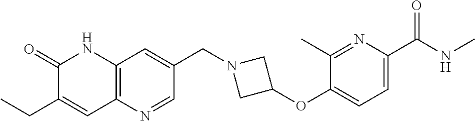 [SMILES: CCc1cc2ncc(CN3CC(Oc4ccc(C(=O)NC)nc4C)C3)cc2[nH]c1=O] | 5-((1-((7-ethyl-6-oxo-5,6- dihydro-1,5-naphthyridin-3- yl)methyl)azetidin-3- yl)oxy)-N,6- dimethylpicolinamide | 408.3 |
| 45 and 46 | 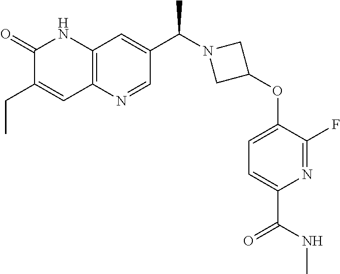 [SMILES: CCc1cc2ncc([C@@H](C)N3CC(Oc4ccc(C(=O)NC)nc4F)C3)cc2[nH]c1=O] | (enantiomer 1 or 2)-5-((1-(1-(7-ethyl-6- oxo-5,6-dihydro-1,5- naphthyridin-3-yl) ethyl)azetidin- 3-yl)oxy)-6-fluoro-N- methylpicolinamide | 426.3 426.2 |
| | 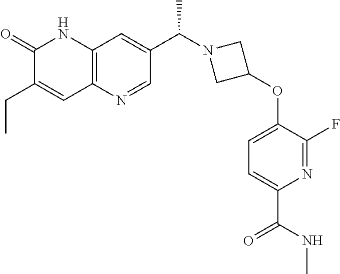 [SMILES: CCc1cc2ncc([C@H](C)N3CC(Oc4ccc(C(=O)NC)nc4F)C3)cc2[nH]c1=O] | | |
| 47 and 48 | 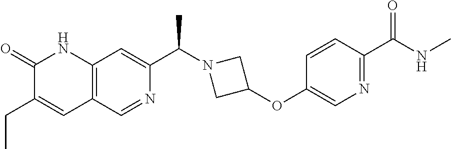 [SMILES: CCc1cc2cnc([C@@H](C)N3CC(Oc4ccc(C(=O)NC)nc4)C3)cc2[nH]c1=O] | (enantiomer 1 or 2)-5-((1-(1-(3-ethyl-2- oxo-1,2-dihydro-1,6- naphthyridin-7- yl)ethyl)azetidin- 3-yl)oxy)-N- methylpicolinamide | 408.2 408.2 |
| | 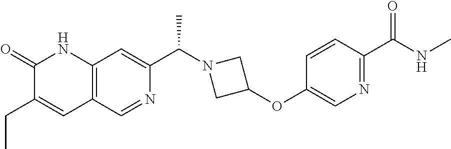 [SMILES: CCc1cc2cnc([C@H](C)N3CC(Oc4ccc(C(=O)NC)nc4)C3)cc2[nH]c1=O] | | |
| 49 and 50 | 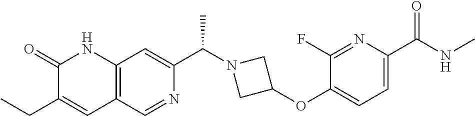 [SMILES: CCc1cc2cnc([C@H](C)N3CC(Oc4ccc(C(=O)NC)nc4F)C3)cc2[nH]c1=O] | (enantiomer 1 or 2)-5-((1-(1-(3-ethyl-2- oxo-1,2-dihydro-1,6- naphthyridin-7-yl) ethyl)azetidin-3-yl) oxy)-6-fluoro-N- methylpicolinamide | 426.0 426.0 |
| | 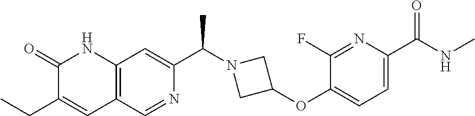 [SMILES: CCc1cc2cnc([C@@H](C)N3CC(Oc4ccc(C(=O)NC)nc4F)C3)cc2[nH]c1=O] | | |
| 51 | 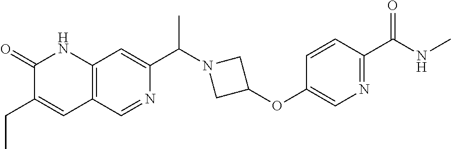 [SMILES: CCc1cc2cnc(C(C)N3CC(Oc4ccc(C(=O)NC)nc4)C3)cc2[nH]c1=O] | 5-((1-(1-(3-ethyl-2-oxo-1,2- dihydro-1,6-naphthyridin- 7-yl)ethyl)azetidin-3-yl) oxy)-N-methylpicolinamide | 408.2 |
| 52 and 53 | 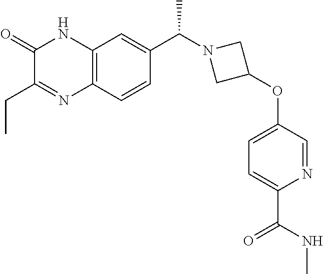 [SMILES: CCc1nc2ccc([C@H](C)N3CC(Oc4ccc(C(=O)NC)nc4)C3)cc2[nH]c1=O] | (enantiomer 1 or 2)-5-((1-(1-(2-ethyl-3- oxo-3,4-dihydroquinoxalin- 6-yl)ethyl)azetidin-3-yl) oxy)-N-methylpicolinamide | 408.2 408.2 |
| | 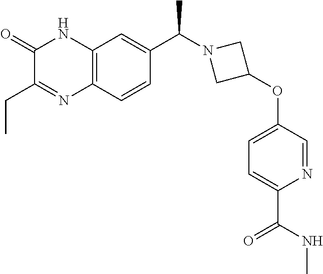 [SMILES: CCc1nc2ccc([C@@H](C)N3CC(Oc4ccc(C(=O)NC)nc4)C3)cc2[nH]c1=O] | | |
| 54 | 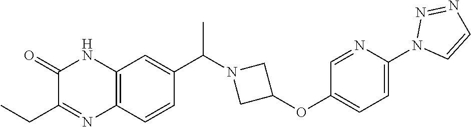 [SMILES: CCc1nc2ccc(C(C)N3CC(Oc4ccc(-n5ccnn5)nc4)C3)cc2[nH]c1=O] | 7-(1-(3-((6-(1H-1,2,3- triazol-1-yl)pyridin-3-yl) oxy)azetidin-1-yl)ethyl)-3- ethylquinoxalin-2(1H)- one | 418.1 |
| 55 | 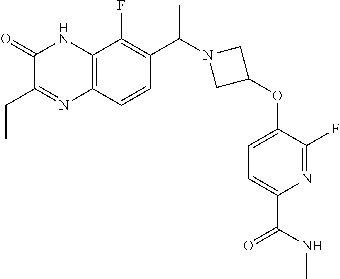 [SMILES: CCc1nc2ccc(C(C)N3CC(Oc4ccc(C(=O)NC)nc4F)C3)c(F)c2[nH]c1=O] | 5-((1-(1-(2-ethyl-5-fluoro- 3-oxo-3,4-dihydroquin oxalin-6-yl)ethyl)azetidin- 3-yl)oxy)-6-fluoro-N- methylpicolinamide | 444.2 |
| 56 and 57 | 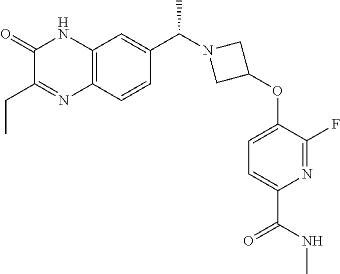 [SMILES: CCc1nc2ccc([C@H](C)N3CC(Oc4ccc(C(=O)NC)nc4F)C3)cc2[nH]c1=O] | (enantiomer 1 or 2)-5-((1-(1-(2-ethyl-3-oxo- 3,4-dihydroquinoxalin- 6-yl)ethyl)azetidin-3-yl) oxy)-6-fluoro-N- methylpicolinamide | 426.3 426.3 |
| | 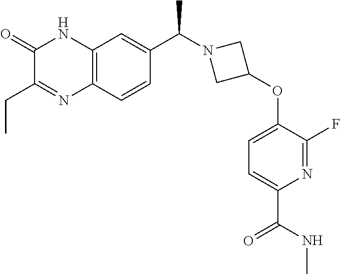 [SMILES: CCc1nc2ccc([C@@H](C)N3CC(Oc4ccc(C(=O)NC)nc4F)C3)cc2[nH]c1=O] | | |
| 58 | 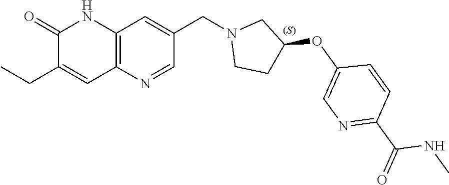 | (S)-5-((1-((7-ethyl-6-oxo- 5,6-dihydro-1,5-naphthyridin- 3-yl)methyl)pyrrolidin- 3-yl)oxy)-N- methylpicolinamide | 408.2 |
| 59 | 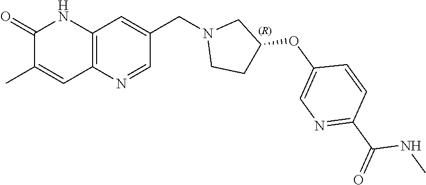 | (R)-N-methyl-5-((1-((7- methyl-6-oxo-5,6-dihydro- 1,5-naphthyridin-3-yl) methyl)pyrrolidin-3-yl) oxy)picolinamide | 394.3 |
| 60 and 61 | 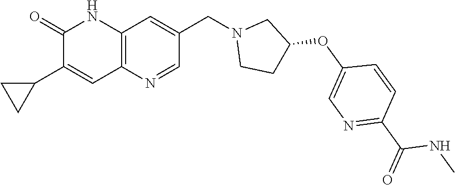 [SMILES: CNC(=O)c1ccc(O[C@@H]2CCN(Cc3cnc4cc(C5CC5)c(=O)[nH]c4c3)C2)cn1] | (enantiomer 1 or 2)-5-((1-((7-cyclopropyl- 6-oxo-5,6-dihydro-1,5- naphthyridin-3-yl)methyl) pyrrolidin-3-yl)oxy)-N- methylpicolinamide | 420.2 420.2 |
| | 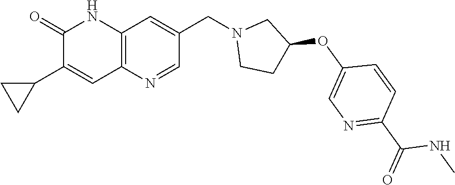 [SMILES: CNC(=O)c1ccc(O[C@H]2CCN(Cc3cnc4cc(C5CC5)c(=O)[nH]c4c3)C2)cn1] | | |
| 62 | 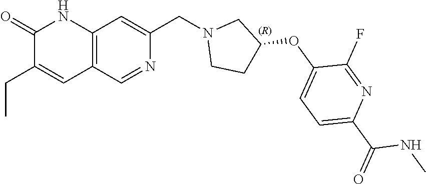 | (R)-5-((1-((3-ethyl-2-oxo- 1,2-dihydro-1,6-naphthyridin- 7-yl)methyl)pyrrolidin- 3-yl)oxy)-6-fluoro-N- methylpicolinamide | 426.2 |
| 63 | 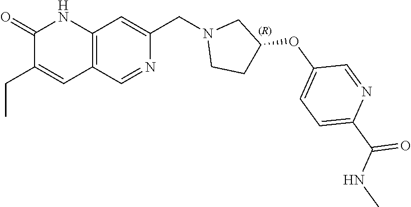 | (R)-5-((1-((3-ethyl-2-oxo- 1,2-dihydro-1,6-naphthyridin- 7-yl)methyl)pyrrolidin- 3-yl)oxy)-N- methylpicolinamide | 408.2 |

To minimize the risks of off-target effects, it is desirable for drug molecules to possess selectivity for a specific target. Advantageously, the compounds of Formula I possess selectivity for PARP1 over PARP2. In an embodiment, there is provided a compound of Formula I having 10-fold selectivity for PARP1 over PARP2. In an embodiment, there is provided a compound of Formula I having 100-fold selectivity for PARP1 over PARP2.

### (II) Pharmaceutical Composition

In another aspect, the present invention provides a pharmaceutical composition comprising a compound of formula I or a pharmaceutically acceptable salt. The composition further comprising at least one pharmaceutically acceptable diluent, excipient or inert carrier. Preferably, the composition is for use in the treatment of cancer.

In one particular variation, the composition is a solid formulation adapted for oral administration. In another particular variation, the composition is a liquid formulation adapted for oral administration. In yet another particular variation, the composition is a tablet. In still another particular variation, the composition is a liquid formulation adapted for parenteral administration.

The present invention also provides a pharmaceutical composition comprising a compound according to anyone of the above embodiments and variations, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

### (III) Method of Treatment

In another aspect, the present invention provides a method of treatment comprising administration of a therapeutically effective amount of compound thereof to a patient in need. Preferably, the patient in need has cancer. Preferably, the cancer is deficient in HR dependent DNA DSB repair pathway. More preferably, the cancer cells have a BRCA1 or BRCA2 deficient phenotype, or the cancer cells are deficient in BRCA1 or BRCA2. More preferably, the cancer is selected from anyone of breast, ovary, pancreas, prostate, hematological, gastrointestinal, and lung cancer.

In another aspect, the present invention provides a method of treatment or prophylaxis of diseases and conditions in which inhibition of PARP1 is beneficial comprising administration of a therapeutically effective amount of compound thereof to a patient in need. Preferably, the patient in need has cancer. More preferably, the patient is heterozygous for a mutation in a gene encoding a component of the HR dependent DNA DSB repair pathway, or the patient is heterozygous for a mutation in a gene encoding a component of the HR dependent DNA DSB repair pathway.

### (IV) Articles of Manufacture

In another aspect, the present invention provides use of the compound compound in the manufacture of a medicament for use in the treatment of diseases or conditions in which inhibition of PARP1 is beneficial. In some embodiments, the cancer is breast, ovary, pancreas, prostate, hematological, gastrointestinal such as gastric and colorectal, or lung cancer. In embodiments, the cancer is breast, ovary, pancreas or prostate cancer.

### (V) General Definitions

Unless the context requires otherwise, throughout the present disclosure and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment disclosed herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

A prefix such as “Cᵤ₋ᵥ” or (Cᵤ-Cᵥ) indicates that the following group has from u to v carbon atoms. For example, “C₁₋₆ alkyl” indicates that the alkyl group has from 1 to 6 carbon atoms.

“Alkyl” refers to a straight or branched chain hydrocarbon radical consisting of carbon and hydrogen atoms, which is saturated, having from one to twelve carbon atoms (C₁₋₁₂ alkyl), in certain embodiments one to eight carbon atoms (C₁₋₈ alkyl) or one to six carbon atoms (C₁₋₆ alkyl), or one to four carbon atoms (C₁₋₄ alkyl), or one to three carbon atoms (C₁₋₃ alkyl), and which is attached to the rest of the molecule by a single bond, e.g., methyl, ethyl, n-propyl, 1-methylethyl (iso-propyl), n-butyl, 1-methylpropyl (sec-butyl), 2-methylpropyl (iso-butyl), 1,1-dimethylethyl (t-butyl), n-pentyl, hexyl, 3-methylhexyl, 2-methylhexyl, and the like.

“Fused” refers to a carbocyclic, heterocyclic, aromatic, or heteroaromatic ring structure described herein which is connected to an existing ring structure in the compounds disclosed herein via two adjacent atoms that are shared by the fused ring structure and the existing ring structure.

“Halo” or “halogen” refers to bromo, chloro, fluoro or iodo.

“Haloalkyl” refers to an alkyl group, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like.

“Alkoxy” means an oxygen moiety having a further alkyl substituent. The alkoxy groups of the present invention can be optionally substituted.

“Cycloalkyl” means a saturated monocyclic, bicyclic, spirocyclic or bridged carbocyclic ring, having a specified number of carbon atoms. Examples of cycloalkyl include cyclopropyl, cyclobutyl, cyclopentyl, cyclohexyl, cycloheptyl, and the like. In one embodiment of the present invention, cycloalkyl is selected from: cyclopropane, cyclobutane and cyclohexane. In another embodiment, cycloalkyl is cyclopropane, cyclobutane or cyclopentane. In another embodiment, cycloalkyl is cyclopropane or cyclobutane. In another embodiment, cycloalkyl is cyclopropane. In another embodiment, cycloalkyl is cyclobutane. In another embodiment, cycloalkyl is cyclopentane. In another embodiment, cycloalkyl is cyclohexane. In another embodiment, cycloalkyl is cycloheptane.

“Oxo” refers to the ═O substituent.

In this specification, unless otherwise stated, the term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In this specification, unless otherwise stated, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician.

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the avoidance of doubt, reference herein to “treatment” includes reference to curative, palliative and prophylactic treatment, and to the administration of a medicament for use in such treatment.

The compounds of Formula I may form stable pharmaceutically acceptable acid or base salts, and in such cases administration of a compound as a salt may be appropriate.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

The compounds of Formula I may have more than one chiral center, and it is to be understood that the application encompasses all individual stereoisomers, enantiomers and diastereoisomers and mixtures thereof. Thus, it is to be understood that, insofar as the compounds of Formula I can exist in optically active or racemic forms by virtue of one or more asymmetric carbon atoms, the application includes in its definition any such optically active or racemic form which possesses the above-mentioned activity. The present application encompasses all such stereoisomers having activity as herein defined.

It is also to be understood that certain compounds of Formula I, and pharmaceutically salts thereof, can exist in solvated as well as unsolvated forms such as, for example, hydrated and anhydrous forms. It is to be understood that the compounds herein encompass all such solvated forms. For the sake of clarity, this includes both solvated (e.g., hydrated) forms of the free form of the compound, as well as solvated (e.g., hydrated) forms of the salt of the compound.

Formula I as described herein is intended to encompass all isotopes of its constituent atoms. For example, FI (or hydrogen) includes any isotopic form of hydrogen including ¹H, ²H (D), and ³H (T); C includes any isotopic form of carbon including ¹²C, ¹³C, and ¹⁴C; O includes any isotopic form of oxygen including ¹⁶O, ¹⁷O and ¹⁸O; N includes any isotopic form of nitrogen including ¹³N, ¹⁴N and ¹⁵N. It is to be understood that the present application encompasses all such isotopic forms.

The compounds of Formula I, or pharmaceutically acceptable salts thereof, will normally be administered via the oral route in the form of pharmaceutical preparations comprising the active ingredient or a pharmaceutically acceptable salt or solvate thereof, or a solvate of such a salt, in a pharmaceutically acceptable dosage form. Depending upon the disorder and patient to be treated, the compositions may be administered at varying doses.

The pharmaceutical formulations of the compound of Formula I described above may be prepared for oral administration, particularly in the form of tablets or capsules, and especially involving technologies aimed at furnishing colon-targeted drug release.

The pharmaceutical formulations of the compound of Formula I described above may conveniently be administered in unit dosage form and may be prepared by anyone of the methods well-known in the pharmaceutical art, for example as described in Remington's Pharmaceutical Sciences, 17th ed., Mack Publishing Company, Easton, PA., (1985).

Pharmaceutical formulations suitable for oral administration may comprise one or more physiologically compatible carriers and/or excipients and may be in solid or liquid form. Tablets and capsules may be prepared with binding agents, fillers, lubricants and/or surfactants, such as sodium lauryl sulfate. Liquid compositions may contain conventional additives such as suspending agents, emulsifying agents and/or preservatives. Liquid compositions may be encapsulated in, for example, gelatin to provide a unit dosage form. Solid oral dosage forms include tablets, two-piece hard shell capsules and soft elastic gelatin (SEG) capsules. Such two-piece hard shell capsules may be made for example by filling a compound of Formula (I) into a gelatin or hydroxypropyl methylcellulose (HPMC) shell.

A dry shell formulation typically comprises of about 40% to 60% w/w concentration of gelatin, about a 20% to 30% concentration of plasticizer (such as glycerin, sorbitol or propylene glycol) and about a 30% to 40% concentration of water. Other materials such as preservatives, dyes, opacifiers and flavours also may be present. The liquid fill material comprises a solid drug that has been dissolved, solubilized or dispersed (with suspending agents such as beeswax, hydrogenated castor oil or polyethylene glycol 4000) or a liquid drug in vehicles or combinations of vehicles such as mineral oil, vegetable oils, triglycerides, glycols, polyols and surface-active agents.

Suitable daily doses of the compounds of Formula I, or a pharmaceutically acceptable salt thereof, in therapeutic treatment of humans are about 0.0001-100 mg/kg body weight.

Oral formulations are preferred, particularly tablets or capsules which may be formulated by methods known to those skilled in the art to provide doses of the active compound in the range of 0.1 mg to 1000 mg.

### EXAMPLES

The invention is further illustrated and explained by referring to the following examples. It should be noted that, the following examples are merely illustrative but not aims to limit the scope of the invention in any manner.

### Example 1: Preparation of 5-((1-((7-ethyl-6-oxo-5,6-dihydro-1,5-naphthyridin-3-yl)methyl)azetidin-3-yl)oxy)-3-fluoro-N-methylpicolinamide (Compound 2)

### Step 1: Methyl 3-fluoro-5-(4,4,5,5-tetramethyl-1,3,2-dioxaborolan-2-yl)pyridine-2-carboxylate

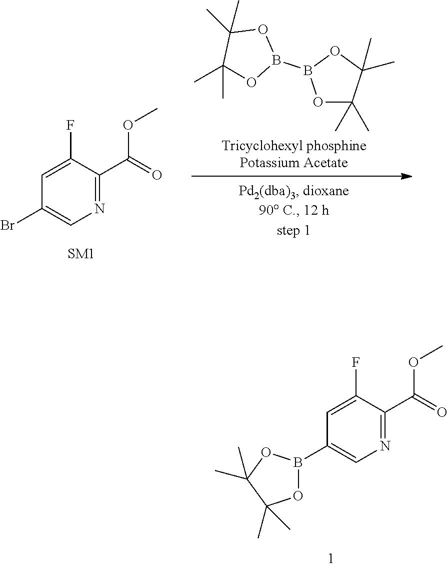

A solution of methyl 5-bromo-3-fluoropyridine-2-carboxylate (4.00 g, 17.1 mmol, 1.0 equiv), bis(pinacolato)diboron (6.51 g, 25.6 mmol, 1.5 equiv), potassium acetate (2.52 g, 25.6 mmol, 1.50 equiv), Pd₂(dba)₃ (626 mg, 0.68 mmol, 0.04 equiv) and tricyclohexylphosphane (383 mg, 1.37 mmol, 0.08 equiv) in dioxane (40 mL) was stirred overnight at 90° C. under N₂ atmosphere. The resulting mixture was extracted with EtOAc (3×200 mL). The combined organic layers were washed with brine (10 mL), dried over anhydrous Na₂SO₄, filtered and concentrated. The crude residue was purified by reversed-phase chromatography (Column: XBridge Prep C18 OBD Column, 30*100 mm, 5 m; Mobile Phase A: Water (10 mmol/L NH₄HCO₃), Mobile Phase B: MeCN; Flow rate: 60 mL/min; Gradient: 35% B to 65% B in 7 min, 65% B; Wave Length: 254/220 nm; RT(min): 6.9) to afford methyl 3-fluoro-5-(4,4,5,5-tetramethyl-1,3,2-dioxaborolan-2-yl)pyridine-2-carboxylate (2.50 g, 52% yield).

### Step 2: Methyl 3-fluoro-5-hydroxypyridine-2-carboxylate

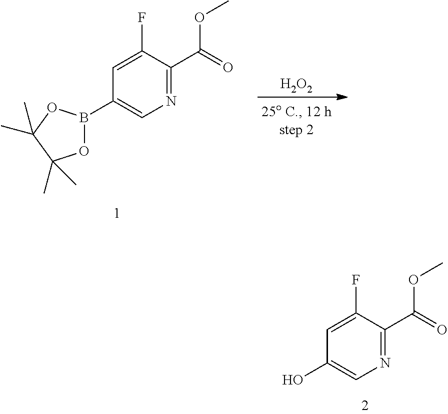

A solution of methyl 3-fluoro-5-(4,4,5,5-tetramethyl-1,3,2-dioxaborolan-2-yl)pyridine-2-carboxylate (2.50 g, 8.89 mmol, 1.0 equiv) in THF (25 mL) was treated with hydrogen peroxide (30% in water, 15 mL) overnight at room temperature. The reaction was then diluted with water (50 mL) and extracted with ethyl acetate (2×50 mL), washed with aqueous 5% sodium thiosulfate solution (50 mL) and brine (50 mL). The organic layer was dried over anhydrous sodium sulfate and concentrated. The residue was purified by silica gel column chromatography, eluted with CH₂Cl₂/MeOH (10:1) to afford methyl 3-fluoro-5-hydroxypyridine-2-carboxylate (1 g, 66% yield).

### Step 3: Methyl 5-{[1-(tert-butoxycarbonyl)azetidin-3-yl]oxy}-3-fluoropyridine-2-carboxylate

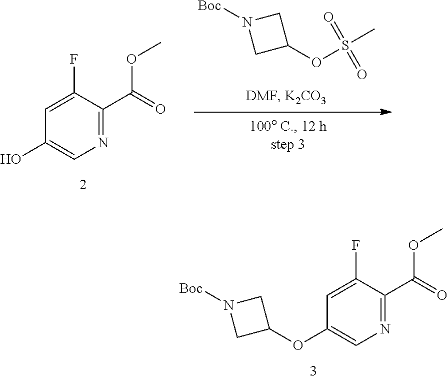 [SMILES: COC(=O)c1ncc(O)cc1F ; CC(C)(C)OC(=O)N1CC(OS(C)(=O)=O)C1 ; COC(=O)c1ncc(OC2CN(C(=O)OC(C)(C)C)C2)cc1F]

A solution of methyl 3-fluoro-5-hydroxypyridine-2-carboxylate (400 mg, 2.34 mmol, 1.0 equiv), tert-butyl 3-(methanesulfonyloxy)azetidine-1-carboxylate (587 mg, 2.34 mmol, 1.0 equiv) and K₂CO₃ (969 mg, 7.01 mmol, 3.0 equiv) in DMF (4 mL) was heated overnight at 80° C. The mixture was cooled down to room temperature and diluted with water (100 mL). The resulting mixture was extracted with EtOAc (3×100 mL). The combined organic layers were washed with water (2×10 mL), dried over anhydrous Na₂SO₄, filtered and concentrated under reduced pressure to afford methyl 5-{[1-(tert-butoxycarbonyl)azetidin-3-yl]oxy}-3-fluoropyridine-2-carboxylate (300 mg, 39% yield) as a yellow oil. The crude product was used in the next step directly without further purification.

### Step 4: tert-Butyl 3-{[5-fluoro-6-(methylcarbamoyl)pyridin-3-yl]oxy}azetidine-1-carboxylate

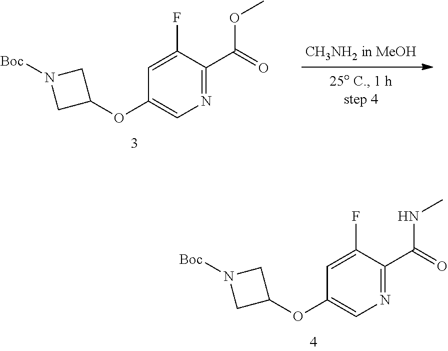 [SMILES: COC(=O)c1ncc(OC2CN(C(=O)OC(C)(C)C)C2)cc1F ; CNC(=O)c1ncc(OC2CN(C(=O)OC(C)(C)C)C2)cc1F]

A solution of methyl 5-{[1-(tert-butoxycarbonyl)azetidin-3-yl]oxy}-3-fluoropyridine-2-carboxylate (250 mg, 0.77 mmol, 1.0 equiv) in MeOH (2.5 mL) was treated with methylamine in MeOH (0.5 mL, 31 wt. %) and the resulting mixture was stirred for 1.5 h at room temperature.. The reaction was acidified to pH 5 with HCl (0.2 M) and concentrated under reduced pressure. The residue was purified by reverse flash chromatography (column, C18 silica gel; mobile phase, MeCN in Water (0.1% FA), 10% to 50% gradient in 10 min) to afford tert-butyl 3-{[5-fluoro-6-(methylcarbamoyl)pyridin-3-yl]oxy}azetidine-1-carboxylate (150 mg, 60% yield) as a light yellow oil.

### Step 5: 5-(Azetidin-3-yloxy)-3-fluoro-N-methylpyridine-2-carboxamide

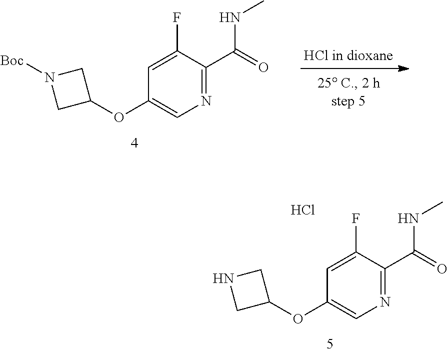 [SMILES: CNC(=O)c1ncc(OC2CN(C(=O)OC(C)(C)C)C2)cc1F ; CNC(=O)c1ncc(OC2CNC2)cc1F ; Cl]

A solution of tert-butyl 3-{[5-fluoro-6-(methylcarbamoyl)pyridin-3-yl]oxy}azetidine-1-carboxylate (150 mg, 0.46 mmol, 1.0 equiv) in a solution of HCl in 1,4-dioxane (3 mL, 4.0 M) was stirred for 2 h at room temperature. The resulting mixture was concentrated under reduced pressure to afford 5-(azetidin-3-yloxy)-3-fluoro-N-methylpyridine-2-carboxamide hydrochloride (100 mg, 96% yield)

### Step 6: 5-((1-((7-ethyl-6-oxo-5,6-dihydro-1,5-naphthyridin-3-yl)methyl)azetidin-3-yl)oxy)-3-fluoro-N-methylpicolinamide

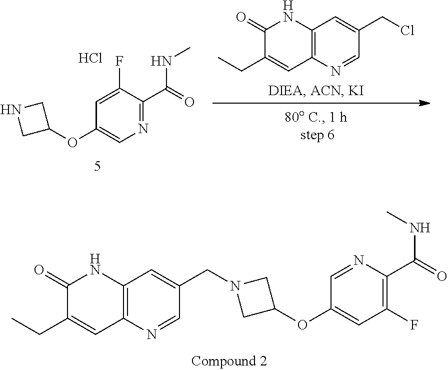

A solution of 7-(chloromethyl)-3-ethyl-1H-1,5-naphthyridin-2-one (100 mg, 0.45 mmol, 1.0 equiv), 5-(azetidin-3-yloxy)-3-fluoro-N-methylpyridine-2-carboxamide hydrochloride (121 mg, 0.54 mmol, 1.2 equiv), potassium iodide (14.9 mg, 0.09 mmol, 0.2 equiv) and DIPEA (391 μL, 2.25 mmol, 5.0 equiv) in MeCN (1 mL) was stirred for 2 h at 80° C. The mixture was concentrated under reduced pressure and then purified by Prep-HPLC (MeCN in Water (0.1% FA), 10% to 50% gradient in 10 min) to afford 5-((1-((7-ethyl-6-oxo-5,6-dihydro-1,5-naphthyridin-3-yl)methyl)azetidin-3-yl)oxy)-3-fluoro-N-methylpicolinamide (36.8 mg, 20% yield).

LC-MS (ES⁺) m/z: 412.35 (M+H)⁺

¹H NMR (400 MHz, DMSO-d₆) δ 11.86 (s, 1H), 8.45 (m, 1H), 8.36 (m, 1H), 8.13 (m, 1H), 7.74 (s, 1H), 7.56 (m, 1H), 7.40 (m, 1H), 5.04 (m, 1H), 3.82-3.78 (m, 4H), 3.29-3.11 (m, 2H), 2.75 (m, 3H), 2.59-2.53 (m, 2H), 1.18 (t, 3H).

### Example 2: Preparation of: (R)-5-((1-(1-(7-ethyl-6-oxo-5,6-dihydro-1,5-naphthyridin-3-yl)ethyl)azetidin-3-yl)oxy)-N-methylpicolinamide (Compound 4), and (S)-5-((1-(1-(7-ethyl-6-oxo-5,6-dihydro-1,5-naphthyridin-3-yl)ethyl)azetidin-3-yl)oxy)-N-methylpicolinamide (Compound 5)

### Step 1: tert-Butyl 3-[(6-cyanopyridin-3-yl)oxy]azetidine-1-carboxylate

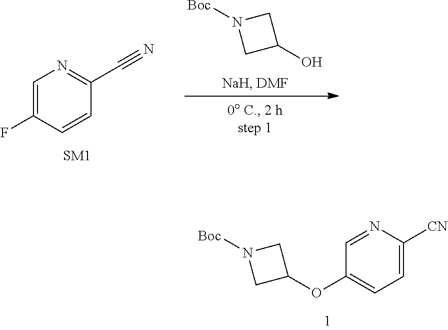 [SMILES: N#Cc1ccc(F)cn1 ; CC(C)(C)OC(=O)N1CC(O)C1 ; CC(C)(C)OC(=O)N1CC(Oc2ccc(C#N)nc2)C1]

A solution of tert-butyl 3-hydroxyazetidine-1-carboxylate (1.70 g, 9.83 mmol, 1.0 equiv) in DMF (10 mL) was treated with NaH (60%) (655 mg, 16.4 mmol, 2.0 equiv) for 30 min at 0° C. followed by the addition of 5-fluoropyridine-2-carbonitrile (1.00 g, 8.19 mmol, 1.0 equiv) in DMF (10 mL) dropwise at 0° C. The reaction was stirred for 2 h at room temperature. The reaction was quenched with water (50 mL) at 0° C. The resulting mixture was extracted with EtOAc (2×50 mL). The combined organic layers were washed with water (10 mL), dried over anhydrous Na₂SO₄, filtered and concentrated under reduced pressure to afford tert-butyl 3-[(6-cyanopyridin-3-yl)oxy]azetidine-1-carboxylate (1.00 g, 44% yield). The crude product was used in the next step directly without further purification.

### Step 2: 5-{[1-(tert-Butoxycarbonyl)azetidin-3-yl]oxy}pyridine-2-carboxylic acid

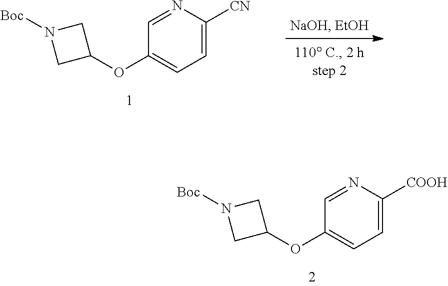 [SMILES: CC(C)(C)OC(=O)N1CC(Oc2ccc(C#N)nc2)C1 ; CC(C)(C)OC(=O)N1CC(Oc2ccc(C(=O)O)nc2)C1]

A solution of tert-butyl 3-[(6-cyanopyridin-3-yl)oxy]azetidine-1-carboxylate (1.00 g, 3.63 mmol, 1.0 equiv) in EtOH (10 mL) was treated with an aqueous solution of NaOH (55 mL, 2.0 M) dropwise at room temperature. The mixture was heated to reflux overnight. The mixture was acidified to pH 5 with conc. HCL. The resulting mixture was extracted with CH₂Cl₂ (2×100 mL). The combined organic layers were dried over anhydrous Na₂SO₄, filtered and concentrated under reduced pressure to afford 5-{[1-(tert-butoxycarbonyl)azetidin-3-yl]oxy}pyridine-2-carboxylic acid (700 mg, 66% yield).

### Step 3: tert-Butyl 3-{[6-(methylcarbamoyl)pyridin-3-yl]oxy}azetidine-1-carboxylate

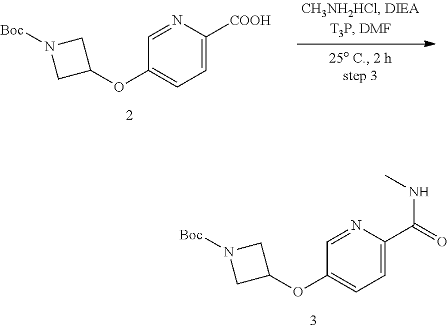 [SMILES: CC(C)(C)OC(=O)N1CC(Oc2ccc(C(=O)O)nc2)C1 ; CNC(=O)c1ccc(OC2CN(C(=O)OC(C)(C)C)C2)cn1]

A solution of 5-{[1-(tert-butoxycarbonyl)azetidin-3-yl]oxy}pyridine-2-carboxylic acid (700 mg, 2.38 mmol, 1.0 equiv) and methylamine hydrochloride (208 mg, 3.09 mmol, 1.3 equiv) in DMF (7.00 mL) was treated with DIPEA (1.36 mL, 7.85 mmol, 3.3 equiv) at −15 T followed by the addition of T₃P (1.97 g, 6.18 mmol, 2.6 equiv) in DMF (7.00 mL) dropwise at −15° C. The resulting mixture was stirred for 2 h at room temperature. Into the resulting mixture was added saturated sodium carbonate solution and stirred for 15 min at room temperature. The precipitated solid was collected by filtration and washed with water (2×5 mL). The crude product was purified by reversed-phase chromatography (Column: XBridge Prep C18 OBD Column, 30*100 mm, 5 μm; Mobile Phase A: Water (10 mmol/L NH₄HCO₃), Mobile Phase B: MeCN; Flow rate: 60 mL/min; Gradient: 35% B to 65% B in 7 min)to afford tert-butyl 3-{[6-(methylcarbamoyl)pyridin-3-yl]oxy}azetidine-1-carboxylate (500 mg, 68% yield).

### Step 4: 5-(Azetidin-3-yloxy)-N-methylpyridine-2-carboxamide

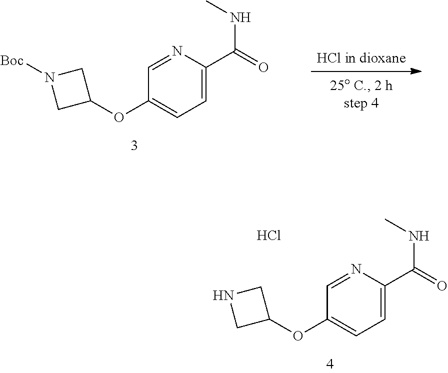 [SMILES: CNC(=O)c1ccc(OC2CN(C(=O)OC(C)(C)C)C2)cn1 ; CNC(=O)c1ccc(OC2CNC2)cn1 ; Cl]

A solution of tert-butyl 3-{[6-(methylcarbamoyl)pyridin-3-yl]oxy}azetidine-1-carboxylate (500 mg, 1.63 mmol, 1.0 equiv) in a solution of HCl in 1,4-dioxane (5 mL, 4.0 M) was stirred for 2 h at room temperature. The resulting mixture was concentrated under reduced pressure to afford 5-(azetidin-3-yloxy)-N-methylpyridine-2-carboxamide,HCl salt (250 mg, 74% yield). The crude product was used in the next step directly without further purification.

### Step 5: 5-((1-(1-(7-ethyl-6-oxo-5,6-dihydro-1,5-naphthyridin-3-yl)ethyl)azetidin-3-yl)oxy)-N methylpicolinamide (Compound 43)

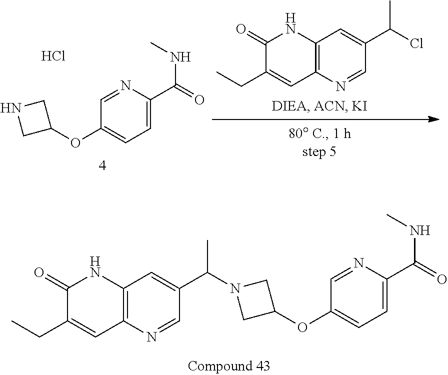

A solution of 7-(1-chloroethyl)-3-ethyl-1H-1,5-naphthyridin-2-one (100 mg, 0.42 mmol, 1.0 equiv), 5-(azetidin-3-yloxy)-N-methylpyridine-2-carboxamide hydrochloride (87.5 mg, 0.42 mmol, 1.0 equiv), potassium iodide (14.0 mg, 0.084 mmol, 0.2 equiv) and DIPEA (368 μL, 2.11 mmol, 5.0 equiv) in MeCN (1.0 mL) was stirred for 2 h at 80° C. The resulting mixture was concentrated under reduced pressure. The crude product was purified by Prep-HPLC (MeCN in Water (10 mmol/L NH₄HCO₃), 10% to 50% gradient in 10 min; detector, UV 254 nm) to afford 5-((1-(1-(7-ethyl-6-oxo-5,6-dihydro-1,5-naphthyridin-3-yl)ethyl)azetidin-3-yl)oxy)-N-methylpicolinamide (77.3 mg, 45% yield).

LC-MS (ES⁺) m/z: 408.35 (M+H)⁺

¹H NMR (400 MHz, DMSO-d₆) δ 11.86 (s, 1H), 8.45 (m, 1H), 8.36 (m, 1H), 8.18 (s, 1H), 8.13 (m, 1H), 7.74 (s, 1H), 7.56 (m, 1H), 7.40 (m, 1H), 5.04 (m, 1H), 3.87-3.78 (m, 1H), 3.58-3.54 (m, 2H), 3.19 (m, 1H), 3.16 (m, 1H), 2.75 (m, 3H), 2.57-2.52 (m, 2H), 1.18 (m, 6H).

### Step 6

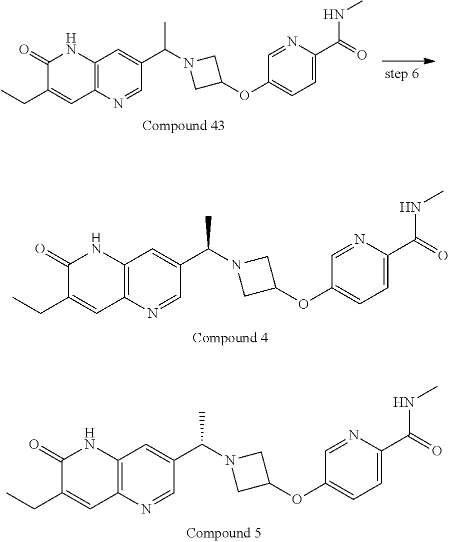

The mixture of enantiomers (40 mg) was resolved by chiral SFC (Column: CHIRAL ART Amylose-SA, 2*25 cm, 5 μm; Mobile Phase A: MtBE (10 mM NH₃-MeOH), Mobile Phase B: MeOH; Flow rate: 20 mL/min; Gradient: 50% B for 40 min; Wave Length: 220/242 nm; RT1(min): 13.02; RT2(min): 29.36; Sample Solvent: MeOH:DCM=1:1; Injection Volume: 1 mL; Number Of Runs: 2) to afford to afford: (R)-5-((1-(1-(7-ethyl-6-oxo-5,6-dihydro-1,5-naphthyridin-3-yl)ethyl)azetidin-3-yl)oxy)-N-methylpicolinamide (Compound 4) (16.9 mg, 42.3% yield).

LC-MS (ES⁺) m/z: 408.35 (M+H)⁺

¹H NMR (400 MHz, DMSO-d₆) δ 11.86 (s, 1H), 8.45 (m, 1H), 8.36 (m, 1H), 8.18 (s, 1H), 8.13 (m, 1H), 7.74 (s, 11H), 7.56 (m, 1H), 7.40 (m, 1H), 5.04 (m, 1H), 3.87-3.78 (m, 1H), 3.58-3.54 (m, 2H), 3.19 (m, 1H), 3.16 (m, 1H), 2.75 (m, 3H), 2.57-2.52 (m, 2H), 1.18 (m, 6H).

Chiral analytical SFC conditions for Compound 4:

Column: CHIRALPAK IA-3, 50×3 mm, 1.6 μm

Mobile phase: (MtBE:DCM=1:1)(0.1% DEA):MeOH=80:20

Flow rate: 1.67 ml/min

RT=0.92 min; 100%

(S)-5-((1-(1-(7-ethyl-6-oxo-5,6-dihydro-1,5-naphthyridin-3-yl)ethyl)azetidin-3-yl)oxy)-N-methylpicolinamide (Compound 5) (14.8 mg, 37.0% yield).

LC-MS (ES⁺) m/z: 408.35 (M+H)⁺

¹H NMR (400 MHz, DMSO-d₆) δ 11.86 (s, 1H), 8.45 (m, 1H), 8.36 (m, 1H), 8.18 (s, 1H), 8.13 (m, 1H), 7.74 (s, 1H), 7.56 (m, 1H), 7.40 (m, 1H), 5.04 (m, 1H), 3.87-3.78 (m, 1H), 3.58-3.54 (m, 2H), 3.19 (m, 1H), 3.16 (m, 1H), 2.75 (m, 3H), 2.57-2.52 (m, 2H), 1.18 (m, 6H).

Chiral analytical SFC conditions for Compound 5:

Column: CHIRALPAK IA-3, 50×3 mm, 1.6 μm

Mobile phase: (MtBE:DCM=1:1)(0.1% DEA):MeOH=80:20

Flow rate: 1.67 ml/min

RT=1.24 min; 99.8%

### Alternative Preparation of Compound 5

### Step 8: (R)—N—((S)-1-(7-ethyl-6-oxo-5,6-dihydro-1,5-naphthyridin-3-yl)ethyl)-2-methylpropane-2-sulfinamide

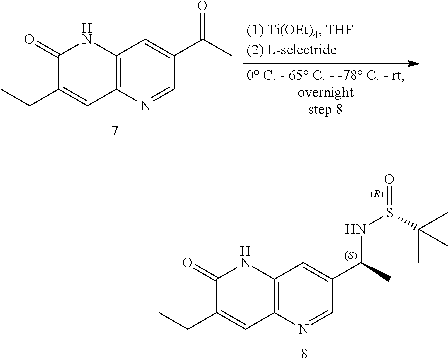

A solution of 7-acetyl-3-ethyl-1,5-naphthyridin-2(1H)-one (150 mg, 0.69 mmol, 1.0 equiv) and (R)-2-methylpropane-2-sulfinamide (92.4 mg, 0.76 mmol, 1.1 equiv) in THF (5 mL) was treated with Ti(OEt)₄ (316 mg, 1.38 mmol, 2.0 equiv) at 0° C. The resulting mixture was stirred for overnight at 65° C. To the above mixture L-selectride (395 mg, 2.08 mmol, 3.0 equiv) was added dropwise at −78° C. The resulting mixture was stirred for additional 15 min at room temperature. The reaction was quenched with MeOH at −50° C. The resulting mixture was concentrated under reduced pressure. The residue was dissolved in DMSO (2 mL). The crude product was purified by Prep-HPLC (column, C18 silica gel; mobile phase, MeCN in Water (10 mmol/L NH₄HCO₃), 10% to 50% gradient in 10 min; detector, UV 254 nm) to afford (R)—N—((S)-1-(7-ethyl-6-oxo-5,6-dihydro-1,5-naphthyridin-3-yl)ethyl)-2-methylpropane-2-sulfinamide (80 mg, 36% yield).

### Step 9: (S)-7-(1-aminoethyl)-3-ethyl-1,5-naphthyridin-2(1H)-one

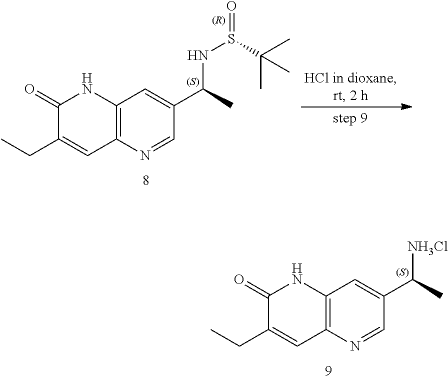

A solution of (R)—N—((S)-1-(7-ethyl-6-oxo-5,6-dihydro-1,5-naphthyridin-3-yl)ethyl)-2-methylpropane-2-sulfinamide (80 mg, 0.24 mmol, 1.0 equiv) in MeOH (1 mL) was treated with hydrogen chloride in 1,4-dioxane (4.0 M, 0.5 mL) at room temperature. After the reaction mixture was stirred for 2 h at room temperature, the reaction was concentrated under reduced pressure to give crude (S)-7-(1-aminoethyl)-3-ethyl-1,5-naphthyridin-2(1H)-one hydrochloride (50 mg, 93% yield).

### Step 10: (S)-3-ethyl-7-(1-(3-hydroxyazetidin-1-yl)ethyl)-1,5-naphthyridin-2(1H)-one

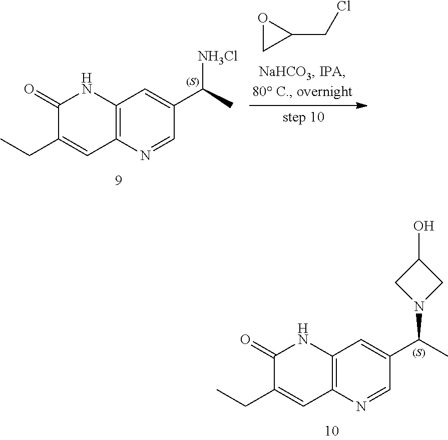

A solution of (S)-7-(1-aminoethyl)-3-ethyl-1,5-naphthyridin-2(1H)-one hydrochloride (50 mg, 0.23 mmol, 1.0 equiv) and epichlorohydrin (21.2 mg, 0.23 mmol, 1.0 equiv) in IPA (1 mL) was treated with NaHCO₃ (29.0 mg, 0.34 mmol, 1.5 equiv) at room temperature. The resulting mixture was stirred overnight at 80° C. The resulting mixture was concentrated under reduced pressure. The crude product was purified by Prep-HPLC (column, C18 silica gel; mobile phase, MeCN in Water (10 mmol/L NH₄HCO₃), 10% to 50% gradient in 10 min; detector, UV 254 nm) to afford (S)-3-ethyl-7-(1-(3-hydroxyazetidin-1-yl)ethyl)-1,5-naphthyridin-2(1H)-one (20 mg, 32% yield).

### Step 11: (S)-5-((1-(1-(7-ethyl-6-oxo-5,6-dihydro-1,5-naphthyridin-3-yl)ethyl)azetidin-3-yl)oxy)picolinonitrile

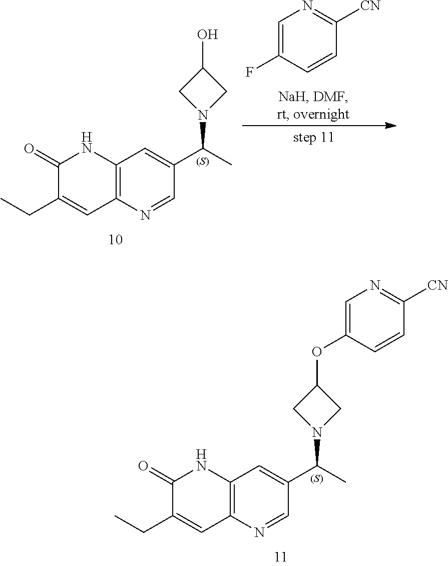

A solution of (S)-3-ethyl-7-(1-(3-hydroxyazetidin-1-yl)ethyl)-1,5-naphthyridin-2(1H)-one (20 mg, 0.073 mmol, 1.0 equiv) in DMF (1 mL) was treated with NaH (60%) (3.51 mg, 0.146 mmol, 2.0 equiv) for 30 minutes at 0° C. followed by the addition of 5-fluoropyridine-2-carbonitrile (13.4 mg, 0.109 mmol, 1.5 equiv) at 0° C. The reaction was quenched with water at 0° C. The crude product was purified by Prep-HPLC (column, C18 silica gel; mobile phase, MeCN in Water (10 mmol/L NH₄HCO₃), 10% to 50% gradient in 10 min; detector, UV 254 nm) to afford (S)-5-((1-(1-(7-ethyl-6-oxo-5,6-dihydro-1,5-naphthyridin-3-yl)ethyl)azetidin-3-yl)oxy)picolinonitrile (10 mg, 36% yield).

LC-MS (ES⁺) m/z: 376.2 (M+H)⁺

### Step 12: (S)-5-((1-(1-(7-ethyl-6-oxo-5,6-dihydro-1,5-naphthyridin-3-yl)ethyl)azetidin-3-yl)oxy)picolinic acid

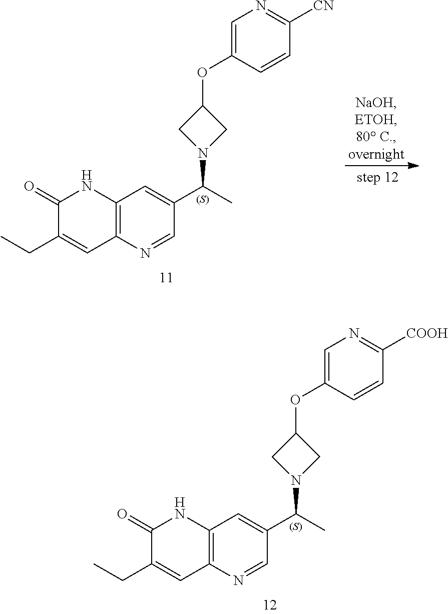

A solution of (S)-5-((1-(1-(7-ethyl-6-oxo-5,6-dihydro-1,5-naphthyridin-3-yl)ethyl)azetidin-3-yl)oxy)picolinonitrile (10 mg, 0.027 mmol, 1 equiv) in EtOH (1 mL) was treated with NaOH (0.01 mL, 10 M) at room temperature. The resulting mixture was stirred overnight at 80° C. The mixture was acidified to pH=5 with HCl (aq.). The crude mixture was concentrated and purified by Prep-HPLC (column, C18 silica gel; mobile phase, MeCN in Water (10 mmol/L NH₄HCO₃), 10% to 50% gradient in 10 min; detector, UV 254 nm) to afford (S)-5-((1-(1-(7-ethyl-6-oxo-5,6-dihydro-1,5-naphthyridin-3-yl)ethyl)azetidin-3-yl)oxy)picolinic acid (5 mg, 48% yield).

LC-MS (ES⁺) m/z: 394.0 (M+H)⁺

### Step 13: (S)-5-((1-(1-(7-ethyl-6-oxo-5,6-dihydro-1,5-naphthyridin-3-yl)ethyl)azetidin-3-yl)oxy)-N-methylpicolinamide (compound 5)

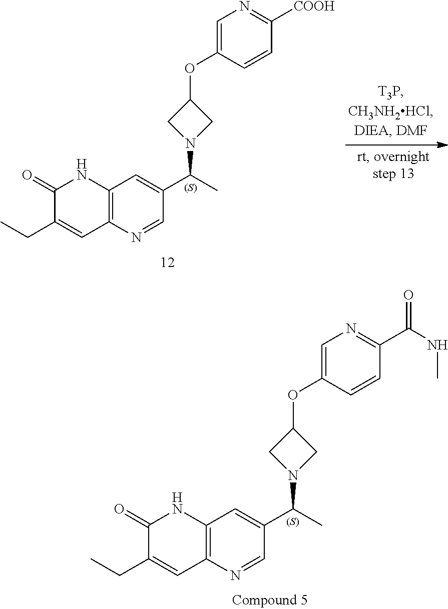

To a solution of (S)-5-((1-(1-(7-ethyl-6-oxo-5,6-dihydro-1,5-naphthyridin-3-yl)ethyl)azetidin-3-yl)oxy)picolinic acid (5 mg, 0.013 mmol, 1.0 equiv) and CH₃NH₂·HCl (0.94 mg, 0.014 mmol, 1.1 equiv) in DMF (1 mL) was added DIEA (8.82 μL, 0.052 mmol, 4.0 equiv). The reaction mixture was stirred for 10 min at room temperature then T3P (1.27 mg, 0.033 mmol, 2.5 equiv) was added at −15° C. The resulting mixture was stirred for 2 h at room temperature. The crude mixture was concentrated and the product was purified by Prep-HPLC (column, C18 silica gel; mobile phase, MeCN in Water (10 mmol/L NH₄HCO₃), 10% to 50% gradient in 10 min; detector, UV 254 nm) to afford (S)-5-((1-(1-(7-ethyl-6-oxo-5,6-dihydro-1,5-naphthyridin-3-yl)ethyl)azetidin-3-yl)oxy)-N-methylpicolinamide (2.0 mg, 39% yield).

LC-MS (ES⁺) m/z: 408.15 (M+H)⁺

¹H NMR (400 MHz, DMSO-d₆) δ 11.81 (s, 1H), 8.57 (q, J=4.8 Hz, 1H), 8.40 (d, J=1.8 Hz, 1H), 8.21 (d, J=2.9 Hz, 1H), 7.93 (d, J=8.7 Hz, 1H), 7.73 (s, 1H), 7.60 (d, J=1.9 Hz, 1H), 7.38 (dd, J=8.7, 2.9 Hz, 1H), 4.95 (p, J=5.5 Hz, 1H), 3.85 (p, J=5.5 Hz, 1H), 3.03-3.65 (dd, J=8.0, 5.0 Hz, 4H), 2.78 (d, J=4.8 Hz, 3H), 2.58-2.51 (m, 2H), 1.23-1.12 (m, 6H).

Chiral analytical SFC:

Column: CHIRALPAK IA-U, 50×3 mm, 1.6 μm

Mobile phase: (MtBE:DCM=2:1)(0.1% DEA):MeOH=70:30

Flow rate: 1.2 ml/min

RT=1.07 min; 97.6%

### Example 3: Preparation of 6-((1-((7-ethyl-6-oxo-5,6-dihydro-1,5-naphthyridin-3-yl)methyl)pyrrolidin-3-yl)amino)-N-methylnicotinamide (Compound 10)

### Step 1: methyl 5-{[1-(tert-butoxycarbonyl)pyrrolidin-3-yl]amino}pyridine-2-carboxylate

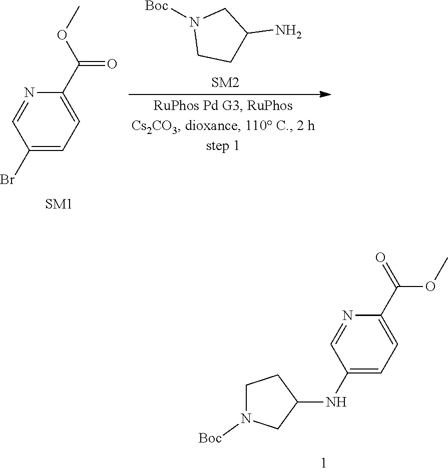 [SMILES: COC(=O)c1ccc(Br)cn1 ; CC(C)(C)OC(=O)N1CCC(N)C1 ; COC(=O)c1ccc(NC2CCN(C(=O)OC(C)(C)C)C2)cn1]

A mixture of 5-bromopyridine-2-carboxylic acid (1.00 g, 4.95 mmol, 1.0 equiv), RuPhos Pd G3 (207 mg, 0.248 mmol, 0.05 equiv), RuPhos (231 mg, 0.495 mmol, 0.1 equiv), Cs₂CO₃ (4.84 g, 14.9 mmol, 3.0 equiv) and tert-butyl-3-aminopyrrolidine-1-carboxylate (921 mg, 4.95 mmol, 1.0 equiv) in 1,4-dioxane (20 mL) was stirred overnight at 110 T under an atmosphere of nitrogen. The reaction was cooled to room temperature and then quenched by the addition of water (20 mL). The resulting mixture was extracted with EtOAc (3×50 mL). The combined organic layers were washed with brine (50 mL), dried over anhydrous Na₂SO₄, filtered, and concentrated under reduced pressure. The residue was purified by reversed-phase chromatography (10-50% MeCN in water (10 mM NH₄HCO₃)) to afford methyl 5-{[1-(tert-butoxycarbonyl)pyrrolidin-3-yl]amino}pyridine-2-carboxylate (1.00 g, 63% yield) as a yellow oil.

### Step 2: tert-butyl 3-{[6-(methylcarbamoyl)pyridin-3-yl]amino}pyrrolidine-1-carboxylate

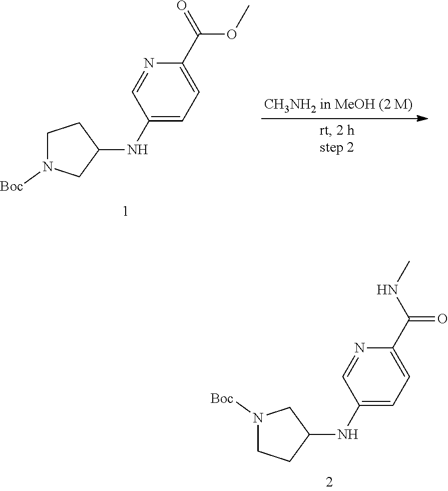 [SMILES: COC(=O)c1ccc(NC2CCN(C(=O)OC(C)(C)C)C2)cn1 ; CNC(=O)c1ccc(NC2CCN(C(=O)OC(C)(C)C)C2)cn1]

A solution of methyl 5-{[1-(tert-butoxycarbonyl) pyrrolidin-3-yl]amino}pyridine-2-carboxylate (1.00 g, 4.36 mmol, 1.0 equiv) and methylamine in MeOH (10 M, 5.0 mL)) was stirred overnight at room temperature under nitrogen atmosphere. The resulting mixture was concentrated under reduced pressure. This resulted in tert-butyl 3-{[6-(methylcarbamoyl) pyridin-3-yl]amino}pyrrolidine-1-carboxylate (800 mg) as a yellow oil which was used in the next step without further purification.

### Step 3: N-methyl-5-(pyrrolidin-3-ylamino)pyridine-2-carboxamide hydrochloride

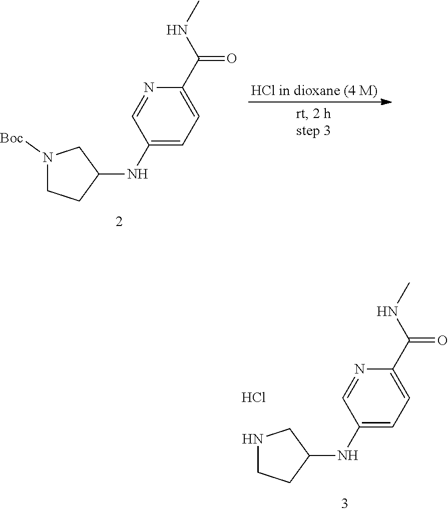 [SMILES: CNC(=O)c1ccc(NC2CCN(C(=O)OC(C)(C)C)C2)cn1 ; CNC(=O)c1ccc(NC2CCNC2)cn1 ; Cl]

A mixture of tert-butyl 3-{[6-(methylcarbamoyl)pyridin-3-yl]amino}pyrrolidine-1-carboxylate (800 mg, 4.06 mmol, 1.0 equiv) and HCl in 1,4-dioxane (4.0 M, 10.2 mL) was stirred for 2 h at room temperature under nitrogen atmosphere. The resulting mixture was concentrated under reduced pressure to afford N-methyl-5-(pyrrolidin-3-ylamino)pyridine-2-carboxamide hydrochloride (600 mg) as a white solid which was used without further purification.

### Step 4: 6-((1-((7-ethyl-6-oxo-5,6-dihydro-1,5-naphthyridin-3-yl)methyl)pyrrolidin-3-yl)amino)-N-methylnicotinamide

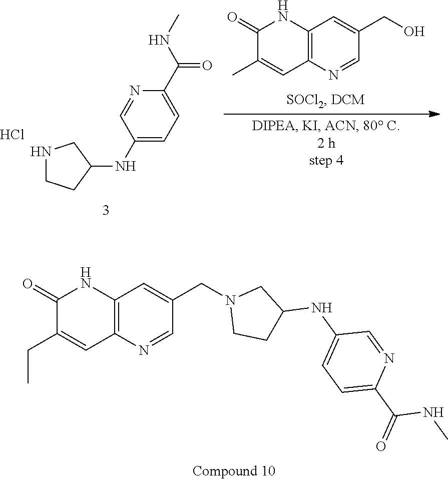

A solution of 3-ethyl-7-(hydroxymethyl)-1H-1,5-naphthyridin-2-one (100 mg, 0.49 mmol, 1.0 equiv) and SOCl₂ (400 μL, 5.51 mmol, 11.2 equiv) in DCM (2 mL) was stirred for 2 h at room temperature under nitrogen atmosphere. The resulting mixture was concentrated under reduced pressure. A mixture of N-methyl-5-(pyrrolidin-3-ylamino)pyridine-2-carboxamide hydrochloride (107 mg, 0.49 mmol, 1.0 equiv), potassium iodide (16.3 mg, 245 μmol, 0.20 equiv) and DIPEA (425 μL, 2.45 mmol, 5.0 equiv) in MeCN (2 mL) was stirred for 2 h at 80° C. under nitrogen atmosphere. The precipitated solids were collected by filtration and washed with water (10 mL). The residue was purified by Prep-HPLC (10% to 31% MeCN in water (0.05% formic acid)) to afford 6-((1-((7-ethyl-6-oxo-5,6-dihydro-1,5-naphthyridin-3-yl)methyl)pyrrolidin-3-yl)amino)-N-meth ylnicotinamide (51.1 mg, 25% yield).

LC-MS (ES⁺) m/z: 407.3 [M+H]⁺

¹H NMR (300 MHz, DMSO-d6): δ 11.87 (s, 1H), 8.40 (m, 1H), 8.30-8.22 (m, 1H), 7.92 (m, 1H), 7.77-7.68 (m, 2H), 7.57 (s, 1H), 6.94 (m, 1H), 6.65 (m, 1H), 3.98 (br s, 1H), 3.76 (s, 2H), 2.89-2.85 (m, 1H), 2.76 (m, 3H), 2.67-2.63 (m, 1H), 2.57-2.53 (m, 2H), 2.45-2.40 (m, 1H), 2.30-2.24 (m, 1H), 1.69-1.58 (m, 1H), 1.18 (m, 3H).

### Example 4: Preparation of (enantiomer 1)-5-((1-((7-ethyl-6-oxo-5,6-dihydro-1,5-naphthyridin-3-yl)methyl)pyrrolidin-3-yl)(methyl) amino)-N-methylpicolinamide (Compound 16), and (enantiomer 2)-5-((1-((7-ethyl-6-oxo-5,6-dihydro-1,5-naphthyridin-3-yl)methyl)pyrrolidin-3-yl)(methyl) amino)-N-methylpicolinamide (Compound 17)

### Step 1: Methyl-5-{[1-(tert-butoxycarbonyl)pyrrolidin-3-yl](methyl)amino}pyridine-2-carboxylate

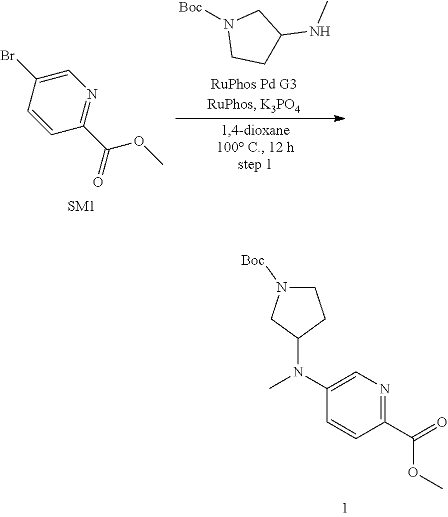 [SMILES: COC(=O)c1ccc(Br)cn1 ; CNC1CCN(C(=O)OC(C)(C)C)C1 ; COC(=O)c1ccc(N(C)C2CCN(C(=O)OC(C)(C)C)C2)cn1]

To a stirred solution of methyl 5-bromopyridine-2-carboxylate (1.50 g, 6.94 mmol, 1.0 equiv) and tert-butyl 3-(methylamino)pyrrolidine-1-carboxylate (1.39 g, 6.94 mmol, 1.0 equiv) in 1,4-dioxane (15 mL) were added RuPhos (323 mg, 0.69 mmol, 0.1 equiv), RuPhos Pd G3 (290 mg, 0.35 mmol, 0.05 equiv), and K₃PO₄ (4.40 g, 20.8 mmol, 3.0 equiv) at 25° C. under N₂ atmosphere. The resulting mixture was stirred for 12 h at 100° C. and then cooled to room temperature. The resulting mixture was filtered and the filter cake washed with 1,4-dioxane (3×5 mL). The filtrate was concentrated under reduced pressure. The residue was purified by reversed-phase chromatography (10-50% MeCN in water (10 mM NH₄HCO₃)) to afford methyl 5-{[1-(tert-butoxycarbonyl)pyrrolidin-3-yl](methyl)amino}pyridine-2-carboxylate (700 mg, 30% yield) as an off-white solid.

### Step 2: tert-Butyl 3-{methyl[6-(methylcarbamoyl)pyridin-3-yl]amino}pyrrolidine-1-carboxylate

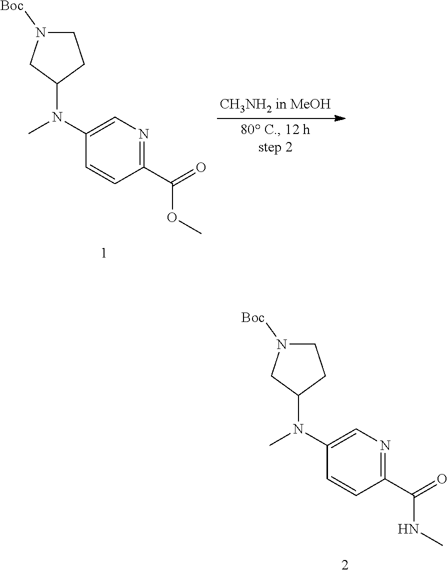 [SMILES: COC(=O)c1ccc(N(C)C2CCN(C(=O)OC(C)(C)C)C2)cn1 ; CNC(=O)c1ccc(N(C)C2CCN(C(=O)OC(C)(C)C)C2)cn1]

A stirred solution of methyl-5-{[1-(tert-butoxy carbonyl)pyrrolidin-3-yl](methyl)amino}pyridine-2-carboxylate (700 mg, 2.09 mmol, 1.0 equiv) and CH₃NH₂ in MeOH (10 M, 6 mL) was stirred overnight at room temperature under nitrogen atmosphere. The resulting mixture was concentrated under reduced pressure to afford tert-butyl 3-{methyl[6-(methylcarbamoyl)pyridin-3-yl]amino}pyrrolidine-1-carboxylate (600 mg, 67% yield) as an off-white solid which was used without further purification.

### Step 3: N-Methyl-5-[methyl(pyrrolidin-3-yl) amino]pyridine-2-carboxamide hydrochloride

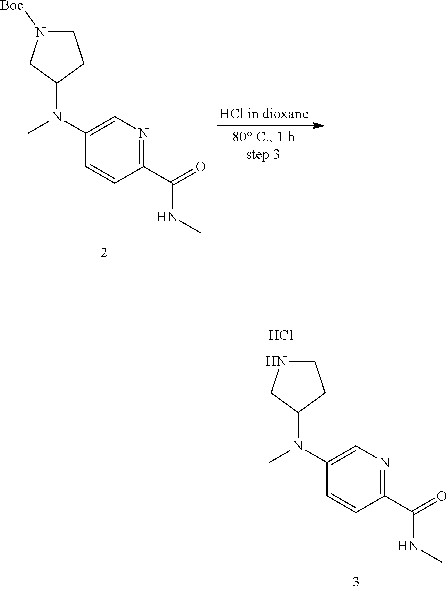 [SMILES: CNC(=O)c1ccc(N(C)C2CCN(C(=O)OC(C)(C)C)C2)cn1 ; Cl ; CNC(=O)c1ccc(N(C)C2CCNC2)cn1]

A stirred solution of tert-butyl 3-{methyl[6-(methylcarbamoyl)pyridin-3-yl]amino}pyrrolidine-1-carboxylate (600 mg, 1.79 mmol, 1.0 equiv) in a solution of HCl in 1,4-dioxane (4.0 M, 5 mL) was stirred at 80 T for 1 hour and then cooled to room temperature. The resulting mixture was concentrated under vacuum. This resulted in N-methyl-5-[methyl(pyrrolidin-3-yl) amino]pyridine-2-carboxamide HCl salt (300 mg, 86% yield) as a white solid which was used without further purification.

### Step 4: 5-({1-[(7-Ethyl-6-oxo-5H-1,5-naphthyridin-3-yl)methyl]pyrrolidin-3-yl}(methyl)amino)-N-methylpyridine-2-carboxamide

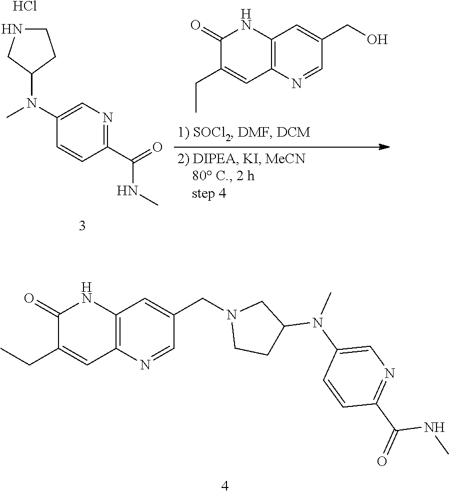 [SMILES: Cl ; CNC(=O)c1ccc(N(C)C2CCNC2)cn1 ; CCc1cc2ncc(CO)cc2[nH]c1=O ; CCc1cc2ncc(CN3CCC(N(C)c4ccc(C(=O)NC)nc4)C3)cc2[nH]c1=O]

SOCl₂ (135 μL, 1.85 mmol, 5.0 equiv) was added dropwise to a stirred solution of 3-ethyl-7-(hydroxymethyl)-1H-1,5-naphthyridin-2-one (75.4 mg, 0.37 mmol, 1.0 equiv) and DMF (2.80 μL, 36.9 μmol, 0.1 equiv) in DCM (2 mL) at room temperature under N₂ atmosphere. The resulting mixture was stirred for 6 h and then concentrated under reduced pressure. potassium iodide (12.3 mg, 73.8 μmol, 0.2 equiv), N-methyl-5-[methyl(pyrrolidin-3-yl)amino]pyridine-2-carboxamide hydrochloride (100 mg, 0.37 mmol, 1.0 equiv) and MeCN (2 mL) were added at room temperature under N₂ atmosphere, followed by DIPEA (386 μL, 2.22 mmol, 6.0 equiv). The resulting mixture was stirred for 2 h at 80° C. and then concentrated under reduced pressure. The residue was purified by Prep-HPLC (10% to 40% MeCN in water (0.1% NH₄HCO₃)) to afford to afford 5-((1-((7-ethyl-6-oxo-5,6-dihydro-1,5-naphthyridin-3-yl)methyl)pyrrolidin-3-yl)(methyl)amino)-N-methylpicolinamide (53.1 mg, 34% yield).

### Step 5: (enantiomer 1)-5-((1-((7-ethyl-6-oxo-5,6-dihydro-1,5-naphthyridin-3-yl)methyl)pyrrolidin-3-yl)(methyl) amino)-N-methylpicolinamide (Compound 16), and (enantiomer 2)-5-((1-((7-ethyl-6-oxo-5,6-dihydro-1,5-naphthyridin-3-yl)methyl)pyrrolidin-3-yl)(methyl) amino)-N-methylpicolinamide (Compound 17)

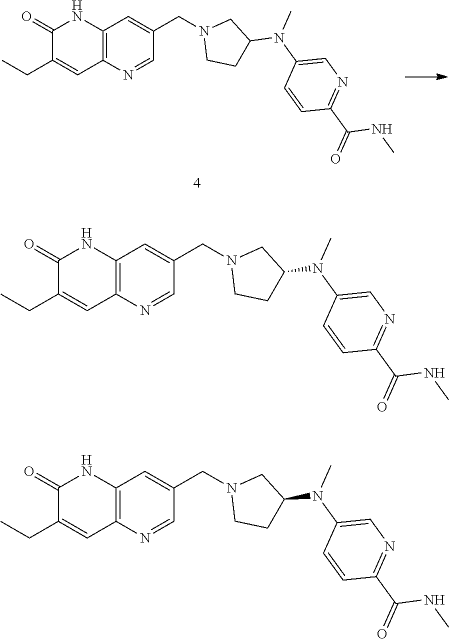

The enantiomers of 5-({1-[(7-ethyl-6-oxo-5H-1,5-naphthyridin-3-yl)methyl]pyrrolidin-3-yl}(methyl)amino)-N-meth ylpyridine-2-carboxamide (50 mg) were resolved by chiral SFC (column: CHIRALPAK IA-3, 4.6×50 mm, 3 μm; mobile phase: 1:1 MTBE (0.1% diethylamine)/MeOH) to afford (enantiomer 1)-5-((1-((7-ethyl-6-oxo-5,6-dihydro-1,5-naphthyridin-3-yl)methyl)pyrrolidin-3-yl)(methyl)amino)-N-methylpicolinamide (13.1 mg, 26% yield) and (enantiomer 2)-5-((1-((7-ethyl-6-oxo-5,6-dihydro-1,5-naphthyridin-3-yl)methyl)pyrrolidin-3-yl)(methyl)amino)-N-methylpicolinamide, (14.9 mg, 30% yield). Absolute configuration of the chiral center for each isolated enantiomer is unknown.

Data for (enantiomer 1)-5-((1-((7-ethyl-6-oxo-5,6-dihydro-1,5-naphthyridin-3-yl)methyl)pyrrolidin-3-yl)(methyl)amino)-N-methylpicolinamide (Compound 16):

LC-MS (ES⁺) m/z: 421.3 [M+H]⁺

¹H NMR (400 MHz, DMSO-d₆) δ 11.89 (s, 1H), 8.41 (m, 1H), 8.30 (m, 1H), 8.11 (m, 1H), 7.84-7.71 (m, 2H), 7.63 (m, 1H), 7.23 (m, 1H), 4.62 (m, 1H), 3.79 (m, 1H), 3.64 (m, 1H), 2.97 (s, 3H), 2.90 (m, 1H), 2.77 (m, 3H), 2.72 (m, 1H), 2.60-2.52 (m, 3H), 2.38-2.20 (m, 2H), 1.80-1.68 (m, 1H), 1.18 (m, 3H).

Chiral analytical SFC conditions for Compound 16:

Column: CHIRALPAK IA-3, 50×4.6 mm, 3.0 μm

Mobile phase: MtBE (0.1% DEA):MeOH=50:50

Flow rate: 1.67 ml/min

RT=4.51 min; 100%

Data for (enantiomer 2)-5-((1-((7-ethyl-6-oxo-5,6-dihydro-1,5-naphthyridin-3-yl)methyl)pyrrolidin-3-yl)(methyl)amino)-N-methylpicolinamide (Compound 17):

LC-MS (ES⁺) m/z: 421.3 [M+H]⁺

¹H NMR (400 MHz, DMSO-d₆) δ 11.89 (s, 1H), 8.41 (m, 1H), 8.30 (m, 1H), 8.11 (m, 1H), 7.84-7.71 (m, 2H), 7.63 (m, 1H), 7.23 (m, 1H), 4.62 (m, 1H), 3.79 (m, 1H), 3.64 (m, 1H), 2.97 (s, 3H), 2.90 (m, 1H), 2.77 (m, 3H), 2.72 (m, 1H), 2.60-2.52 (m, 3H), 2.38-2.20 (m, 2H), 1.80-1.68 (m, 1H), 1.18 (m, 3H).

Chiral analytical SFC conditions for Compound 17:

Column: CHIRALPAK IA-3, 50×4.6 mm, 3.0 μm

Mobile phase: MtBE (0.1% DEA):MeOH=50:50

Flow rate: 1.67 ml/min

RT=1.95 min; 100%

### Example 5: Preparation of (enantiomer 1)-5-((1-(1-(7-ethyl-6-oxo-5,6-dihydro-1,5-naphthyridin-3-yl)ethyl)azetidin-3-yl)oxy)-6-fluoro-N-methylpicolinamide (Compound 45), and (enantiomer 2)-5-((1-(1-(7-ethyl-6-oxo-5,6-dihydro-1,5-naphthyridin-3-yl)ethyl)azetidin-3-yl)oxy)-6-fluoro-N-methylpicolinamide (Compound 46)

### Step 1: tert-Butyl 3-[(6-chloro-2-fluoropyridin-3-yl)oxy]azetidine-1-carboxylate

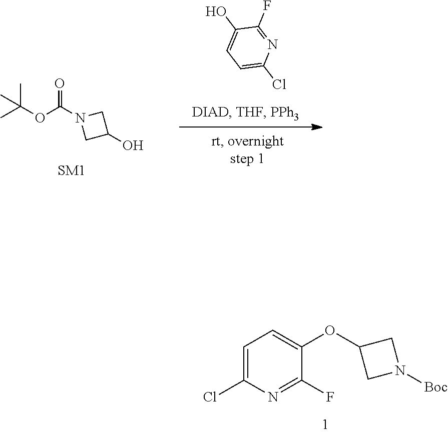 [SMILES: CC(C)(C)OC(=O)N1CC(O)C1 ; Oc1ccc(Cl)nc1F ; CC(C)(C)OC(=O)N1CC(Oc2ccc(Cl)nc2F)C1]

To a stirred solution of tert-butyl 3-hydroxyazetidine-1-carboxylate (2.00 g, 11.5 mmol, 1.0 equiv), 6-chloro-2-fluoropyridin-3-ol (1.70 g, 11.5 mmol, 1.0 equiv) and PPh₃ (3.63 g, 13.9 mmol, 1.2 equiv) in THF (20 mL) was added DIAD (2.72 mL, 13.9 mmol, 1.2 equiv) at 0° C. under nitrogen atmosphere. The resulting mixture was stirred overnight at room temperature. The resulting mixture was filtered and the filter cake was washed with DCM (3×3 mL). The filtrate was concentrated under reduced pressure and then purified by reversed-phase flash chromatography (column, C₁₈; mobile phase, MeCN in Water (10 mmol/L NH₄HCO₃), 10% to 70% gradient in 15 min; detector, UV 254 nm) to afford tert-butyl 3-[(6-chloro-2-fluoropyridin-3-yl)oxy]azetidine-1-carboxylate (1.00 g, 29% yield) as a light yellow solid.

### Step 2: tert-Butyl 3-[(6-cyano-2-fluoropyridin-3-yl)oxy]azetidine-1-carboxylate

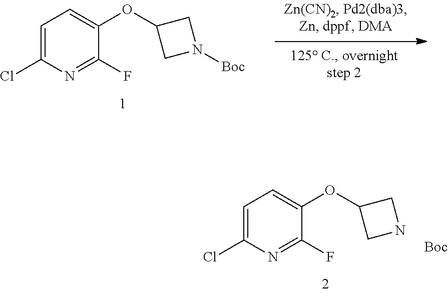 [SMILES: CC(C)(C)OC(=O)N1CC(Oc2ccc(Cl)nc2F)C1 ; Fc1nc(Cl)ccc1OC1CNC1 ; "Boc"]

To a stirred solution of tert-butyl 3-[(6-chloro-2-fluoropyridin-3-yl)oxy]azetidine-1-carboxylate (1.00 g, 3.30 mmol, 1.0 equiv) and Zn(CN)₂ (0.39 g, 3.30 mmol, 1.0 equiv) in DMAc (10 mL) were added dppf (0.18 g, 0.33 mmol, 0.1 equiv) and Pd₂(dba)₃ (0.15 g, 0.16 mmol, 0.05 equiv) at room temperature under nitrogen atmosphere. The resulting mixture was stirred overnight at 125° C. The resulting mixture was filtered, and the filter cake washed with DMAc (3×5 mL). The filtrate was concentrated under reduced pressure and then purified by prep-HPLC (Column: XSelect CSH Prep C₁₈ OBD Column, 19*150 mm, 5 μm; Mobile Phase A: Water (0.05% FA), Mobile Phase B: ACN; Flow rate: 25 mL/min; Gradient: 10% B to 31% B in 7 min, 31% B; Wave Length: 254/220 nm) to afford tert-butyl 3-[(6-cyano-2-fluoropyridin-3-yl)oxy]azetidine-1-carboxylate (700 mg, 72% yield) as an off-white solid.

### Step 3: tert-butyl 3-((6-carbamoyl-2-fluoropyridin-3-yl)oxy)azetidine-1-carboxylate

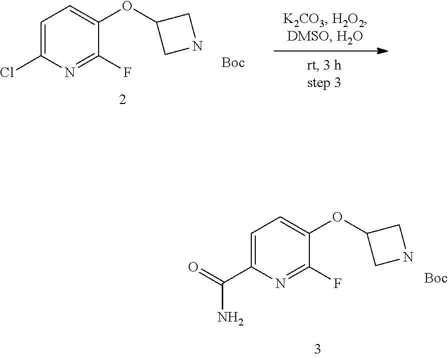

To a stirred solution of tert-butyl 3-[(6-cyano-2-fluoropyridin-3-yl)oxy]azetidine-1-carboxylate (700 mg, 2.38 mmol, 1.0 equiv) and K₂CO₃ (0.13 g, 0.95 mmol, 0.4 equiv) in DMSO (14 mL) was added 30% aqueous H₂O₂ (7 mL) dropwise at room temperature under nitrogen atmosphere. The resulting mixture was stirred for 3 h at room temperature and then diluted with water (30 mL). The precipitated solids were collected by filtration and washed with H₂O (3×5 mL). This resulted in tert-butyl 3-((6-carbamoyl-2-fluoropyridin-3-yl)oxy)azetidine-1-carboxylate (320 mg, 43% yield) as an off-white solid. The crude product was used in the next step without further purification

### Step 4: tert-Butyl 3-{[2-fluoro-6-(methylcarbamoyl)pyridin-3-yl]oxy}azetidine-1-carboxylate

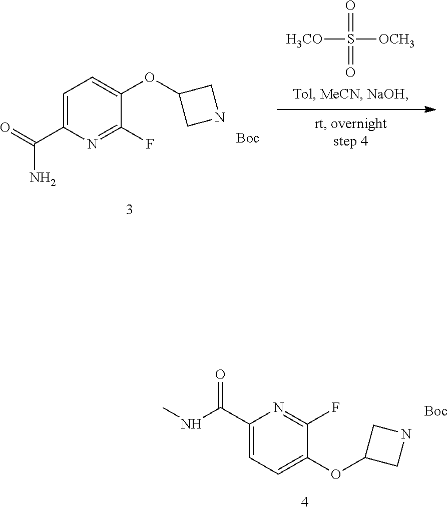

To a stirred solution of tert-butyl 3-[(6-carbamoyl-2-fluoropyridin-3-yl)oxy]azetidine-1-carboxylate (320 mg, 1.03 mmol, 1.0 equiv) and benzyltriethylazanium chloride (234 mg, 1.03 mmol, 1.0 equiv) and dimethyl sulfate (117 μL, 1.23 mmol, 1.2 equiv) in toluene (4.5 mL) and MeCN (4.5 mL) was added NaOH (41.1 mg, 1.03 mmol, 1 equiv) at room temperature under nitrogen atmosphere. The resulting mixture was stirred overnight and then concentrated under vacuum. The residue was purified by prep-HPLC (Column: XSelect CSH Prep C₁₈ OBD Column, 19*150 mm, 5 μm; Mobile Phase A: Water (0.05% FA), Mobile Phase B: ACN; Flow rate: 25 mL/min; Gradient: 10% B to 31% B in 7 min, 31% B; Wave Length: 254/220 nm) to afford tert-butyl 3-{[2-fluoro-6-(methylcarbamoyl)pyridin-3-yl]oxy}azetidine-1-carboxylate (200 mg, 60% yield) as an off-white solid.

### Step 5: 5-(azetidin-3-yloxy)-6-fluoro-N-methylpicolinamide

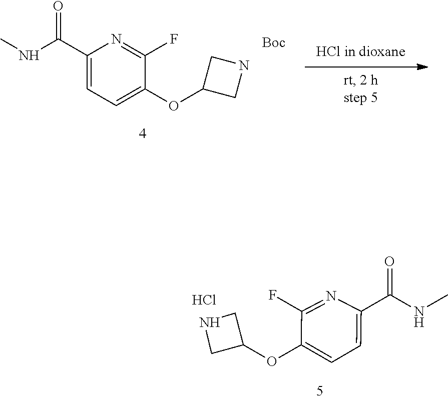

Into a 40 mL vial were added tert-butyl 3-{[2-fluoro-6-(methylcarbamoyl)pyridin-3-yl]oxy}azetidine-1-carboxylate (200 mg, 0.61 mmol, 1.0 equiv) and hydrogen chloride in dioxane (4.0 M, 7 mL) at room temperature. The resulting mixture was stirred for additional 2 h at room temperature. The resulting mixture was concentrated under vacuum. This resulted in 5-(azetidin-3-yloxy)-6-fluoro-N-methylpicolinamide hydrochloride (170 mg) as an off-white solid. The crude product was used in the next step directly without further purification.

### Step 6: 5-((1-(1-(7-ethyl-6-oxo-5,6-dihydro-1,5-naphthyridin-3-yl)ethyl)azetidin-3-yl)oxy)-6-fluoro-N-methylpicolinamide (Compound 21)

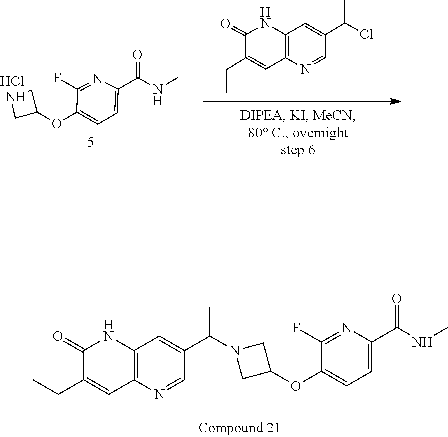 [SMILES: Cl ; CNC(=O)c1ccc(OC2CNC2)c(F)n1 ; CCc1cc2ncc(C(C)Cl)cc2[nH]c1=O ; CCc1cc2ncc(C(C)N3CC(Oc4ccc(C(=O)NC)nc4F)C3)cc2[nH]c1=O]

Into a 20 mL vial were added 5-(azetidin-3-yloxy)-6-fluoro-N-methylpicolinamide hydrochloride (170 mg, 0.75 mmol, 1.7 equiv), 7-(1-chloroethyl)-3-ethyl-1H-1,5-naphthyridin-2-one (105 mg, 0.44 mmol, 1.0 equiv), KI (22.1 mg, 0.13 mmol, 0.3 equiv) and MeCN (8 mL) at room temperature. To the above mixture was added DIEA (425 μL, 2.44 mmol, 5.5 equiv) dropwise at room temperature and the resulting mixture stirred overnight at 80° C. The resulting mixture was concentrated under reduced pressure and the residue purified by Prep-TLC (CH₃CN/H₂O 3:1) to afford 5-((1-(1-(7-ethyl-6-oxo-5,6-dihydro-1,5-naphthyridin-3-yl)ethyl)azetidin-3-yl)oxy)-6-fluoro-N-methylpicolinamide (Compound 21) (19.1 mg, 10% yield).

LC-MS (ES⁺) m/z: 426.2 (M+H)⁺

¹H NMR (400 MHz, DMSO-d₆) δ 11.85 (s, 1H), 8.48 (q, J=4.7 Hz, 1H), 8.42 (d, J=1.8 Hz, 1H), 7.85 (d, J=8.2 Hz, 1H), 7.75 (s, 1H), 7.65-7.51 (m, 2H), 5.00 (t, J=5.5 Hz, 1H), 3.83 (d, J=7.0 Hz, 1H), 3.59 (td, J=6.6, 4.1 Hz, 2H), 3.18 (dd, J=8.1, 5.0 Hz, 1H), 3.08 (dd, J=8.1, 5.0 Hz, 1H), 2.78 (d, J=4.8 Hz, 3H), 2.58-2.53 (m, 2H), 1.24-1.16 (m, 6H).

### Step 7: (enantiomer 1)-5-((1-(1-(7-ethyl-6-oxo-5,6-dihydro-1,5-naphthyridin-3-yl)ethyl)azetidin-3-yl)oxy)-6-fluoro-N-methylpicolinamide (Compound 45) and (enantiomer 2)-5-((1-(1-(7-ethyl-6-oxo-5,6-dihydro-1,5-naphthyridin-3-yl)ethyl)azetidin-3-yl)oxy)-6-fluoro-N-methylpicolinamide (Compound 46)

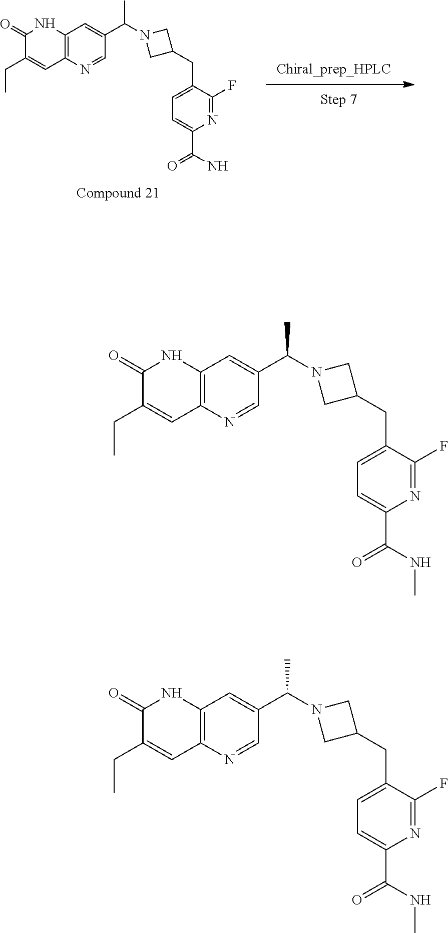 [SMILES: CCc1cc2ncc(C(C)N3CC(Cc4ccc(C(N)=O)nc4F)C3)cc2[nH]c1=O ; CCc1cc2ncc([C@@H](C)N3CC(Cc4ccc(C(=O)NC)nc4F)C3)cc2[nH]c1=O ; CCc1cc2ncc([C@H](C)N3CC(Cc4ccc(C(=O)NC)nc4F)C3)cc2[nH]c1=O]

The enantiomers of 5-((1-(1-(7-ethyl-6-oxo-5,6-dihydro-1,5-naphthyridin-3-yl)ethyl)azetidin-3-yl)oxy)-6-fluoro-N-methylpicolinamide (Compound 21) (18 mg) were separated by SFC (Column: CHIRALPAK IA, 2*25 cm, 5 μm; Mobile Phase A: MtBE:DCM=2:1, Mobile Phase B: MeOH; Flow rate: 20 mL/min; 30% B for 10 min; Wave Length: 330/224 nm; RT1(min): 4.0; RT2(min): 7.0) to afford:

(enantiomer 1)-5-((1-(1-(7-ethyl-6-oxo-5,6-dihydro-1,5-naphthyridin-3-yl)ethyl)azetidin-3-yl)oxy)-6-fluoro-N-methylpicolinamide (Compound 45) (5.6 mg, 31% yield).

LC-MS (ES⁺) m/z: 426.3 (M+H)⁺

1H NMR (400 MHz, DMSO-d6) δ 11.85 (s, 1H), 8.48 (d, J=5.0 Hz, 1H), 8.41 (s, 1H), 7.84 (d, J=8.1 Hz, 1H), 7.73 (s, 1H), 7.64-7.51 (m, 2H), 4.99 (s, 1H), 3.83 (s, 1H), 3.58 (s, 2H), 3.17 (d, J=7.5 Hz, 1H), 3.06 (s, 1H), 2.76 (d, J=4.8 Hz, 3H), 2.61-2.52 (m, 2H), 1.19 (m, 6H).

Chiral analytical SFC conditions for Compound 45:

Column: CHIRALPAK IA-U, 50×3.0 mm, 1.6 μm

Mobile phase: MtBE/DCM (2:1) (0.1% DEA):MeOH=70:30

Flow rate: 1.2 ml/min

RT=0.459 min; 100%

(enantiomer 2)-5-((1-(1-(7-ethyl-6-oxo-5,6-dihydro-1,5-naphthyridin-3-yl)ethyl)azetidin-3-yl)oxy)-6-fluoro-N-methylpicolinamide (Compound 46) (5.7 mg, 32% yield).

LC-MS (ES⁺) m/z: 426.2 (M+H)⁺

1H NMR (400 MHz, DMSO-d6) δ 11.88 (s, 1H), 8.48 (d, J=31.0 Hz, 2H), 8.41 (s, 1H), 7.87 (d, J=8.2 Hz, 1H), 7.77 (s, 1H), 7.61 (d, J=11.1 Hz, 2H), 5.02 (s, 1H), 3.86 (s, 1H), 3.61 (s, 2H), 3.24-3.06 (m, 2H), 2.79 (d, J=4.8 Hz, 3H), 2.58 (t, J=7.2 Hz, 2H), 1.57-0.96 (m, 6H).

Chiral analytical SFC conditions for Compound 46:

Column: CHIRALPAK IA-U, 50×3.0 mm, 1.6 μm

Mobile phase: MtBE/DCM (2:1) (0.1% DEA):MeOH=70:30

Flow rate: 1.2 ml/min

RT=0.703 min; 99.7%

Absolute configuration of the chiral center for each isolated enantiomer is unknown.

### Example 6: Preparation of (enantiomer 1)-5-((1-(1-(3-ethyl-2-oxo-1,2-dihydro-1,6-naphthyridin-7-yl)ethyl)azetidin-3-yl)oxy)-6-fluoro-N-methylpicolinamide (Compound 49) and (enantiomer 2)-5-((1-(1-(3-ethyl-2-oxo-1,2-dihydro-1,6-naphthyridin-7-yl)ethyl)azetidin-3-yl)oxy)-6-fluoro-N-methylpicolinamide (Compound 50)

### Step 6: 7-chloro-3-ethyl-1,6-naphthyridin-2(1H)-one

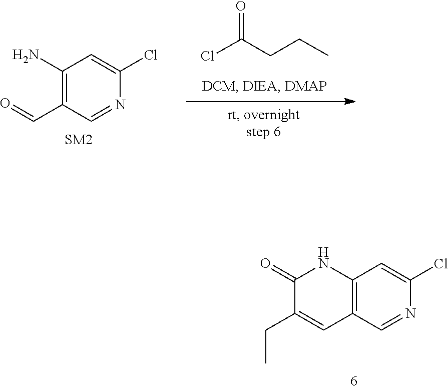

A solution of 4-amino-6-chloropyridine-3-carbaldehyde (10.0 g, 64.0 mmol, 1.0 equiv), DIEA (55.8 mL, 320 mmol, 5.0 equiv), DMAP (1.56 g, 12.8 mmol, 0.20 equiv) in DCM (100 mL) was treated with butyryl chloride (21.9 mL, 211 mmol, 3.30 equiv) for 4 h at 0° C. under nitrogen atmosphere followed by the addition of butyryl chloride (19.7 mL, 190 mmol, 3.0 equiv) dropwise at 0° C. The resulting mixture was stirred overnight at room temperature. The reaction was quenched by the addition of water (100 mL) at room temperature. The precipitated solids were collected by filtration and washed with water (3×50 mL). This resulted in 7-chloro-3-ethyl-1,6-naphthyridin-2(1H)-one (2.40 g, 36% yield) as a white solid. The crude product was used without further purification.

### Step 7: 7-(1-ethoxyvinyl)-3-ethyl-1,6-naphthyridin-2(1H)-one

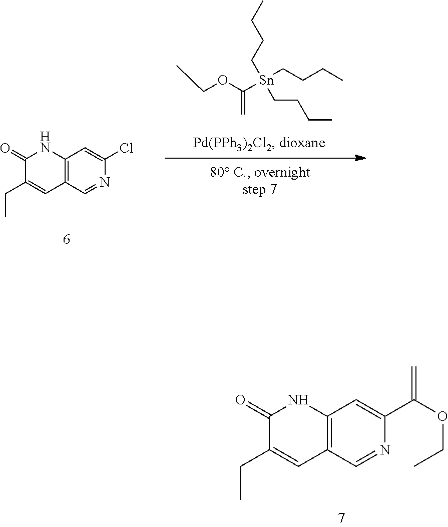 [SMILES: C=[C](OCC)[Sn]([CH2]CCC)([CH2]CCC)[CH2]CCC ; CCc1cc2cnc(Cl)cc2[nH]c1=O ; C=C(OCC)c1cc2[nH]c(=O)c(CC)cc2cn1]

A solution of 7-chloro-3-ethyl-1,6-naphthyridin-2(1H)-one (2.40 g, 11.5 mmol, 1.0 equiv), Pd(PPh₃)₂Cl₂ (0.81 g, 1.15 mmol, 0.1 equiv) and tributyl(1-ethoxyethenyl)stannane (10.4 g, 28.8 mmol, 2.5 equiv) in dioxane (50 mL) was stirred overnight at 110° C. under nitrogen atmosphere. The resulting mixture was used in the next step directly without further purification.

### Step 8: 7-acetyl-3-ethyl-1,6-naphthyridin-2(1H)-one

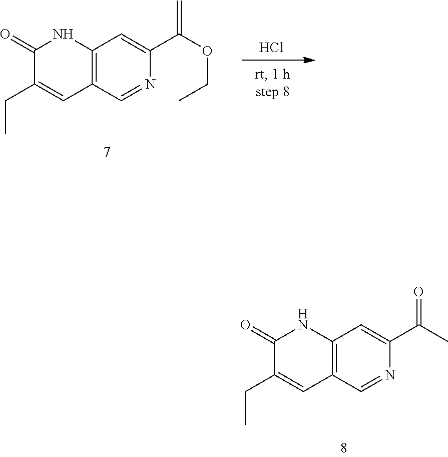 [SMILES: C=C(OCC)c1cc2[nH]c(=O)c(CC)cc2cn1 ; Cl ; CCc1cc2cnc(C(C)=O)cc2[nH]c1=O]

A mixture of 7-(1-ethoxyvinyl)-3-ethyl-1,6-naphthyridin-2(1H)-one (60 mL, mixture) and HCl (2 mL) in dioxane (10 mL) was stirred for 1 h at room temperature under nitrogen atmosphere. The resulting mixture was concentrated under reduced pressure. The residue was purified by reversed-phase flash chromatography (column, C18 silica gel; mobile phase, MeCN in Water (10 mmol/L NH₄HCO₃), 10% to 50% gradient in 10 min; detector, UV 254 nm) to afford 7-acetyl-3-ethyl-1,6-naphthyridin-2(1H)-one (700 mg, 16% yield) as a brown solid.

### Step 9: 3-ethyl-7-(1-hydroxyethyl)-1,6-naphthyridin-2(1H)-one

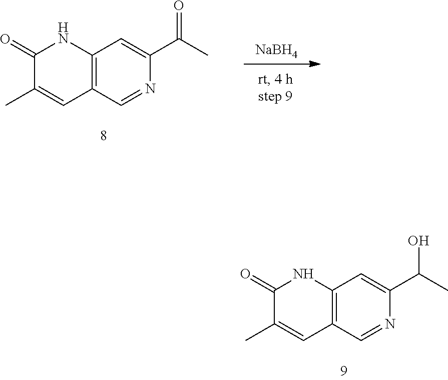

A solution of 7-acetyl-3-ethyl-1,6-naphthyridin-2(1H)-one (700 mg, 3.24 mmol, 1.0 equiv) and NaBH₄ (490 mg, 13.0 mmol, 4.0 equiv) in MeOH (10 mL) was stirred for 4 h at room temperature under nitrogen atmosphere. The resulting mixture was concentrated under reduced pressure and purified by reversed-phase flash chromatography (column, C18 silica gel; mobile phase, MeCN in Water (10 mmol/L NH₄HCO₃), 10% to 50% gradient in 10 min; detector, UV 254 nm) to afford 3-ethyl-7-(1-hydroxyethyl)-1,6-naphthyridin-2(1H)-one (500 mg, 71% yield) as a yellow solid.

### Step 10: 7-1-bromoethyl)-3-ethyl-1,6-naphthyridin-2(1H)-one

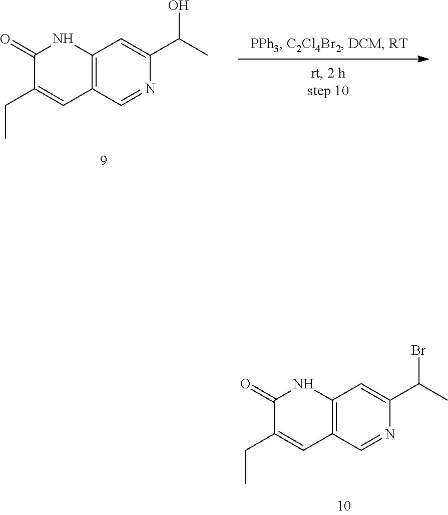 [SMILES: CCc1cc2cnc(C(C)O)cc2[nH]c1=O ; CCc1cc2cnc(C(C)Br)cc2[nH]c1=O]

A solution of 3-ethyl-7-(1-hydroxyethyl)-1,6-naphthyridin-2(1H)-one (500 mg, 2.30 mmol, 1.0 equiv) in DCM (5 mL) was treated with 1,2-dibromo-1,1,2,2-tetrachloroethane (1.64 g, 5.04 mmol, 2.2 equiv) for 20 min at 0 T under nitrogen atmosphere followed by the addition of PPh3 (1.20 g, 4.58 mmol, 2.0 equiv) in DCM (5 mL) dropwise at 0° C. The resulting mixture was stirred for additional 4 h at room temperature. The resulting mixture was concentrated under reduced pressure to afford 7-(1-bromoethyl)-3-ethyl-1,6-naphthyridin-2(1H)-one (400 mg, crude) as a brown solid that was used without further purification.

### Step 11: 5-((1-(1-(3-ethyl-2-oxo-1,2-dihydro-1,6-naphthyridin-7-yl)ethyl)azetidin-3-yl)oxy)-6-fluoro-N-methylpicolinamide

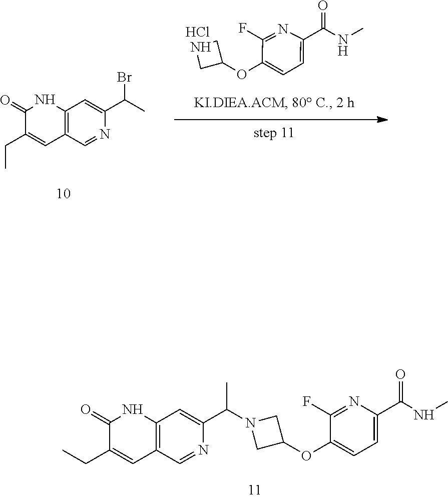 [SMILES: Cl ; CNC(=O)c1ccc(OC2CNC2)c(F)n1 ; CCc1cc2cnc(C(C)Br)cc2[nH]c1=O ; CCc1cc2cnc(C(C)N3CC(Oc4ccc(C(=O)NC)nc4F)C3)cc2[nH]c1=O]

A solution of 7-(1-bromoethyl)-3-ethyl-1,6-naphthyridin-2(1H)-one (400 mg, 1.42 mmol, 1.0 equiv), DIEA (1.24 mL, 7.12 mmol, 5.0 equiv), KI (47.3 mg, 0.285 mmol, 0.20 equiv) and 5-(azetidin-3-yloxy)-6-fluoro-N-methylpicolinamide hydrochloride (320 mg, 1.43 mmol, 1.0 equiv) in MeCN (10 mL) was stirred for 2 h at 80° C. under nitrogen atmosphere. The resulting mixture was concentrated under reduced pressure and the residue purified by reversed-phase flash chromatography (column, C18 silica gel; mobile phase, MeCN in Water (10 mmol/L NH₄HCO₃), 10% to 40% gradient in 10 min; detector, UV 254 nm) to afford 5-((1-(1-(3-ethyl-2-oxo-1,2-dihydro-1,6-naphthyridin-7-yl)ethyl)azetidin-3-yl)oxy)-6-fluoro-N-methylpicolinamide (150 mg, 25% yield).

LC-MS (ES⁺) m/z: 426.3 (M+H)⁺

### Step 12: (enantiomer 1)-5-((1-(1-(3-ethyl-2-oxo-1,2-dihydro-1,6-naphthyridin-7-yl)ethyl)azetidin-3-yl)oxy)-6-fluoro-N-methylpicolinamide (Compound 49) and (enantiomer 2)-5-((1-(1-(3-ethyl-2-oxo-1,2-dihydro-1,6-naphthyridin-7-yl)ethyl)azetidin-3-yl)oxy)-6-fluoro-N-methylpicolinamide (Compound 50)

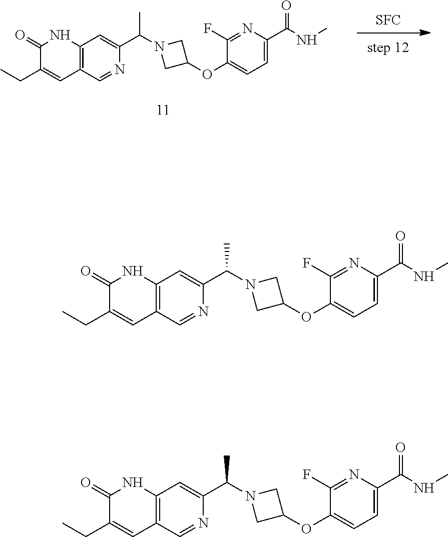

The enantiomers of 5-((1-(1-(3-ethyl-2-oxo-1,2-dihydro-1,6-naphthyridin-7-yl)ethyl)azetidin-3-yl)oxy)-6-fluoro-N-methylpicolinamide (100 mg) were separated by SFC (Column: CHIRAL ART Cellulose-SB, 4.6*100 mm, 3.0 μm; Mobile Phase: MtBE(0.1% DEA): EtOH=80:20; Flow rate: 1.67 mL/min) to afford:

(enantiomer 1)-5-((1-(1-(3-ethyl-2-oxo-1,2-dihydro-1,6-naphthyridin-7-yl)ethyl)azetidin-3-yl)oxy)-6-fluoro-N-methylpicolinamide (Compound 49) (30 mg, 30% yield).

LC-MS (ES⁺) m/z: 426.0 (M+H)⁺

¹H NMR (400 MHz, DMSO-d6): δ 11.92 (s, 1H), 8.71 (s, 1H), 8.48 (d, J=5.0 Hz, 1H), 7.85 (d, J=8.2 Hz, 1H), 7.78 (s, 1H), 7.56 (dd, J=10.2, 8.2 Hz, 1H), 7.23 (s, 1H), 5.02 (t, J=5.4 Hz, 1H), 3.79 (t, J=6.9 Hz, 1H), 3.68 (t, J=7.1 Hz, 1H), 3.57 (q, J=6.4 Hz, 1H), 3.23 (dd, J=8.1, 4.7 Hz, 1H), 3.11 (dd, J=8.3, 4.8 Hz, 1H), 2.77 (d, J=4.7 Hz, 3H), 2.52 (s, 1H), 2.50-2.44 (m, 1H), 1.26-1.12 (m, 6H).

SFC RT=1.993 min; 100%

(enantiomer 2)-5-((1-(1-(3-ethyl-2-oxo-1,2-dihydro-1,6-naphthyridin-7-yl)ethyl)azetidin-3-yl)oxy)-6-fluoro-N-methylpicolinamide (Compound 50) (34 mg, 34% yield).

LC-MS (ES⁺) m/z: 426.0 (M+H)⁺

¹H NMR (400 MHz, DMSO-d6): δ 11.92 (s, 1H), 8.71 (s, 1H), 8.48 (q, J=4.7 Hz, 1H), 7.85 (d, J=8.2 Hz, 1H), 7.78 (s, 1H), 7.56 (dd, J=10.1, 8.2 Hz, 1H), 7.23 (s, 1H), 5.01 (q, J=5.4 Hz, 1H), 3.79 (t, J=7.0 Hz, 1H), 3.68 (t, J=7.2 Hz, 1H), 3.57 (q, J=6.5 Hz, 1H), 3.23 (dd, J=8.2, 4.8 Hz, 1H), 3.11 (dd, J=8.1, 4.8 Hz, 1H), 2.77 (d, J=4.7 Hz, 3H), 2.52 (s,1H), 2.50-2.44 (m, 1H), 1.26-1.12 (m, 6H).

SFC RT=2.353 min; 100%

Absolute configuration of the chiral center for each isolated enantiomer is unknown.

### Example 7 Biological Assays

### FP Assays:

FP experiments were carried out at room temperature using no-binding black 384-well microplates. Recombinant full length PARP1 and PARP2 proteins produced in house were diluted to 20 nM and 60 nM, respectively, with assay buffer (50 mM Tris pH 8, 0.001% Triton X100, 10 mM MgCl₂, and 150 mM NaCl) and incubated for 4 h with an equivalent volume of the 8 nM fluorescent probe diluted with assay buffer. Fluorescence anisotropy of the probe when bound to the proteins was measured in the presence of test compounds or solvent control and the effect on anisotropy determined. Polarization values were read using an Envision plate reader using excitation and emission wavelengths of 590 and 630 nm, respectively. All FP values are expressed as mP units. Inhibition ratio was calculated using readout (mP) following the equation as below:

$$Inhibition ( \% ) = 100 \times ( {mP}_{HC} - {mP}_{sample} ) / ( {mP}_{HC} - {mP}_{LC} ) , \\ where HC and LC represent the high and low control wells , respectively .$$

% inhibition values for different test compound concentrations were calculated and fitted to a four parameter logistic plot in order to determine the IC₅₀ value using XLfit.

The test results are summarized in Table 2.

### Cell Proliferation Assays in DLD1-BRCA2−/− Cell Lines

BRCA2 (−/−) cells cultured in RPMI 1640+10% FBS were harvested and diluted to a density of 1×10⁴ cells/mL and 2×10⁴ cells/mL, respectively. Cells (40 μL/well) were seeded into 384-well cell culture plates. Plates were covered and incubated at 37° C., 5% CO₂ overnight prior to the addition of test compounds or vehicle. The plates were then incubated at 37° C., 5% CO₂ for 7 days. On day 8, the plates were removed from incubator and equilibrated at room temperature for 15 minutes. CellTiter-Glo (40 μL, at 1:1 to culture medium) is added into each well and the plates were placed at room temperature for 30 min. Luminescence was measured using an Envision plate reader. The resultant data were analysed as follows; where LC is culture medium without cells:

$$\% Vehicle = 100 \times ( Lum Test Sample - Lum LC ) / ( Lum HC - Lum LC ) , \\ where LC and HC are the low and high control wells , respectively .$$

% inhibition values for different test compound concentrations were calculated and fitted to a four parameter logistic plot in order to determine the IC₅₀ value using XLfit

The test results are summarized in Table 2.

Assays Method of Caco2 (a-B/B-a)

The apical-to-basolateral (A-B) and basolateral-to-apical (B-A) transport of 5 μM test compounds in HBSS (10 mM HEPES, pH 7.4) was measured across Caco-2 cell monolayers (cells sourced from American Type Culture Collection, Manassas, Virgina). Duplicate incubations were performed at approximately 37° C. for 120 min, with functionality of the test system confirmed using 5 μM propranolol and digoxin as control compounds. Aliquots (50 μL) from both apical and basolateral wells were transferred into two fresh 96-well plates and quenched with acetonitrile solution containing analytical internal standards. Samples were vortexed mixed, centrifuged and a 100 μL aliquot of the resulting supernatant mixed with an equal volume of ultra-pure water prior to analysis by UPLC-MS/MS. The concentrations of test compound and control compounds in the incubation medium of donor and receiver compartments at the beginning and the end of the incubation period were used to calculate the apparent permeability (Papp) from A-B and B-A directions. The efflux ratio (ER) was expressed as Papp_{B-A}/Papp_{A-B}. The integrity of the cell monolayers after 2 hours incubation was confirmed using the marker reagent Lucifer yellow.

The test results are summarized in Table 2.

TABLE 2

| Compound | FP PARP1 | FP PARP2 | BRCA2 (−/−) | Caco₂ (A-B/B-A) |
|---|---|---|---|---|
| No. | IC₅₀ (nM) | IC₅₀ (nM) | cell assay (nM) | 10*−6 cm/s |
| 1 | 4.8 | >10000 | 4.5 | 3.86/22.68 |
| | | | | (ER = 5.87) |
| 2 | 7.7 | >10000 | 288.2 | 1.47/28.27 |
| | | | | (ER = 19.19) |
| 3 | 4.3 | >10000 | 221.3 | — |
| 4 | 10.3 | >10000 | 216 | 4.77/35.73 |
| | | | | (ER = 7.49) |
| 5 | 5.2 | 3122 | 6.9 | 3.39/33.22 |
| | | | | (ER = 9.78) |
| 6 | 7.0 | >10000 | 62.1 | 3.32/30.83 |
| | | | | ER = 9.29) |
| 7 | 7.8 | 7038 | 206.9 | — |
| 8 | 3.8 | >10000 | 51.1 | 2.45/28.82 |
| | | | | (ER = 11.75) |
| 9 | — | — | 14.3 | 3.55/36.54 |
| | | | | (ER = 10.30) |
| 10 | 3.1 | >10000 | 11.5 | 0.57/22.35 |
| | | | | (ER = 39.13) |
| 11 | 4.8 | >10000 | 6.0 | 4.13/40.03 |
| | | | | (ER = 9.69) |
| 12 | — | — | 22.5 | 3.44/35.55 |
| | | | | (ER = 10.32) |
| 13 | — | — | 29.5 | 3.85/30.43 |
| | | | | (ER = 7.91) |
| 14 | 6.6 | >10000 | 48.6 | — |
| 15 | 18.0 | >10000 | 194.7 | — |
| 16 | — | — | >1000 | — |
| 17 | — | — | 194.8 | 3.64/33.29 |
| | | | | (ER = 9.14) |
| 18 | — | — | >1000 | — |
| 19 | 6.1 | >10000 | >1000 | — |
| 20 | 14.77 | >10000 | 38.2 | 11.76/25.39 |
| | | | | (ER = 2.16) |
| 21 | 3.5 | 4813 | 17.2 | — |
| 22 | — | — | >1000 | — |
| 23 | — | — | >1000 | — |
| 24 | 7.3 | >10000 | 541.6 | — |
| 25 | — | — | 299.9 | — |
| 26 | — | — | 191.8 | — |
| 27 | — | — | 355.9 | — |
| 28 | 9.3 | >10000 | 57.6 | 6.32/31.82 |
| | | | | (ER = 5.03) |
| 29 | 9.6 | >10000 | 7.9 | — |
| 30 | 4.4 | >10000 | 256.4 | — |
| 31 | — | — | >1000 | 3.50/30.86 |
| | | | | (ER = 8.81) |
| 32 | — | — | >1000 | — |
| 33 | — | — | >1000 | — |
| 34 | — | — | >1000 | — |
| 35 | — | — | >1000 | — |
| 36 | — | — | >1000 | — |
| 37 | — | — | >1000 | 1.61/30.21 |
| | | | | (ER = 18.78) |
| 38 | — | — | >1000 | — |
| 39 | — | — | >1000 | — |
| 40 | — | — | 181.2 | 4.29/35.68 |
| | | | | (ER = 8.32) |
| 41 | — | — | >1000 | — |
| 42 | — | — | >1000 | 4.57/35.27 |
| | | | | (ER = 7.71) |
| 43 | 5.9 | 3756 | 12.2 | — |
| 44 | 4.4 | >10000 | >1000 | — |
| 45 | 6.4 | 364.7 | 69.3 | 4.18/43.90 |
| | | | | (ER = 10.49) |
| 46 | 4.3 | 2575 | 6.6 | 5.51/44.49 |
| | | | | (ER = 8.07) |
| 47 | 5.8 | >10000 | 26.8 | 4.82/35.60 |
| | | | | (ER = 7.38) |
| 48 | 5.1 | >10000 | 21.9 | 8.96/22.47 |
| | | | | (ER = 2.51) |
| 49 | 14.47 | >10000 | 5.7 | 3.46/29.89 |
| | | | | (ER = 8.64) |
| 50 | 18.11 | >10000 | 7.2 | 3.84/32.1 |
| | | | | (ER = 8.36) |
| 51 | 3.8 | 9609.2 | 15.9 | 5.40/34.56 |
| | | | | (ER = 6.40) |
| 52 | 35.21 | >10000 | 18.9 | 14.92/31.2 |
| | | | | (ER = 2.09) |
| 53 | 24.2 | >10000 | 12.1 | 13.2/33.19 |
| | | | | (ER = 2.51) |
| 54 | 2.12 | — | 38.5 | — |
| 55 | 28.11 | — | 554.3 | 9.92/14.85 |
| | | | | (ER = 1.50) |
| 56 | 11 | — | 10.9 | 8.54/25.76 |
| | | | | (ER = 3.01) |
| 57 | 7.3 | — | 9.1 | 12.25/29.21 |
| | | | | (ER = 2.38) |
| 58 | 44.73 | >10000 | >1000 | — |
| 59 | — | — | 215.3 | — |
| 60 | 64.27 | >10000 | >1000 | — |
| 61 | 6.62 | >10000 | 40.7 | — |
| 62 | — | — | 1.6 | 2.46/31.19 |
| | | | | (ER = 12.66) |
| 63 | 7.03 | >10000 | 5.4 | 5.35/42.55 |
| | | | | (ER = 7.96) |

— Means not tested

### Example 8 Solubility Assays

### Kinetic Solubility, PBS, pH7.4

Kinetic solubility incubations were performed in duplicate at 25° C., 1100 rpm for 2 hours in PBS pH 7.4 containing 300 μM of test compound or the control compound progesterone, prepared in DMSO at 10 mM concentration, in 1.5 mL glass vials in the Eppendorf Thermomixer Comfort plate shaker. After incubation, the samples were filtered, and the filtrate was diluted by 1000-fold with water:acetonitrile 1:1 (v/v) for analysis by UPLC MS/MS to determine the concentration of test compound. The solubility value was calculated by quantification against a standard of known concentration.

The test results are summarized in Table 3.

### Thermodynamic Solubility, FaSSiF

All incubations were performed in duplicate. Test compound or the control compound diclofenac sodium (1.0 mg) were placed into 1.5 mL glass vials. FaSSIF (1000 μL) was added into the vials. The samples were transferred to Eppendorf Thermomixer Comfort plate shaker and shaken at 25° C., 1100 rpm for 24 hours. The samples were then filtered. The filtrate was diluted by 1000-fold with water:acetonitrile 1:1 (v/v) for analysis by UPLC MS/MS to determine the concentration of test compound. The solubility value was calculated by quantification against a standard of known concentration.

The test results are summarized in Table 3.

TABLE 3

| | Kinetic solubility, | Thermodynamic Solubility, |
|---|---|---|
| Compound | PBS, pH 7.4 | FaSSiF, |
| No. | (μM) | μg/mL |
| 1 | 29.38 | 1.77 |
| 4 | 223.12 | 115.18 |
| 5 | 250.15, 281.58* | 106.58, 145.89, 126.09^{#} |

*Test results of two different batches
^{#}Test results of three different batches

What is claimed is:

1. A compound of formula I: 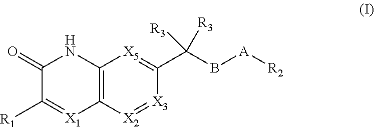
or a pharmaceutically acceptable salt thereof, wherein,
X₁, X₂ or X₃ is independently selected from the group consisting of N or CH;
X₅ is selected from the group consisting of N, CH or CF;
A is —O—;
B is selected from the group consisting of one substituted or unsubstituted 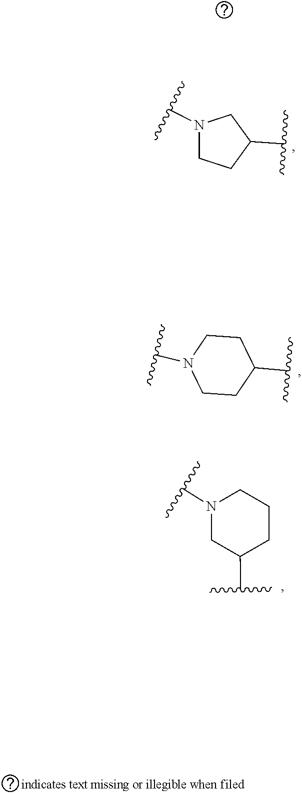 [SMILES: *C1CCN(*)C1 ; *C1CCN(*)CC1 ; *C1CCCN(*)C1]
and the substituted group at any position of ring B is R₅;
R₁ is selected from the group consisting of C₁-C₆ alkyl, C₁-C₆ haloalkyl, C₁-C₆ alkoxy, C₃-C₅ cycloalkyl;
R₂ is 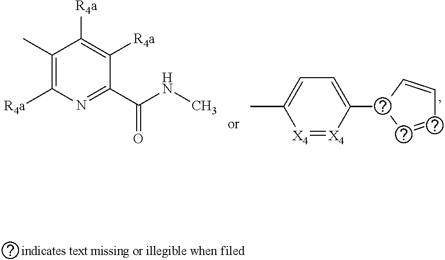
wherein one X₄ is N and one X₄ is CH;
each R₃ is independently selected from the group consisting of H or unsubstituted or substituted —C₁-C₆ alkyl, the substitutes of —C₁-C₆ alkyl is selected from the group consisting of H, —O—CH₃, —CN, —OH, or two R₃ are attached to form a C₃-C₅ cycloalkyl;
each R₄ₐ is independently selected from the group consisting of H, CN, halogen, C₁-C₆ alkyl, —O-alkyl, C₁-C₆ haloalkyl, or —C₁-C₆ alkoxy;
R₅ is selected from the group consisting of H, C₁-C₆ alkyl, ═O, —(CH₂)₁₋₃OH, or halogen.

2. The compound according to claim 1, wherein the formula I is 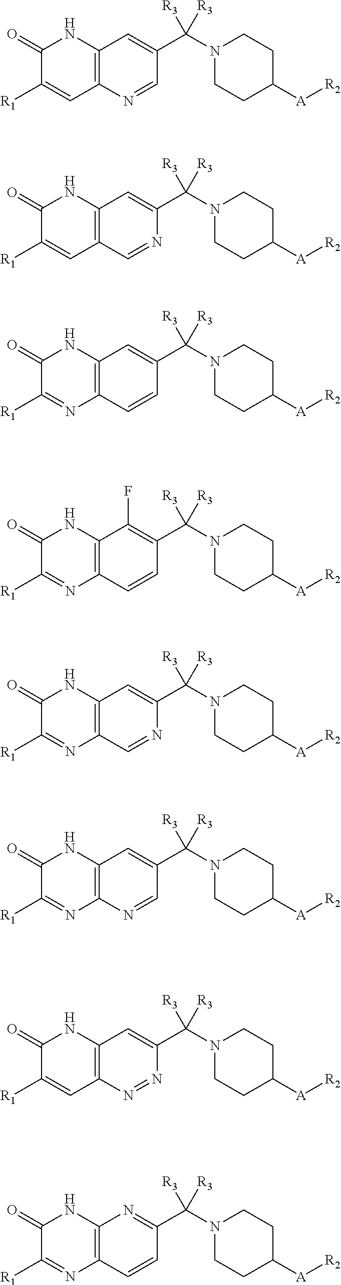 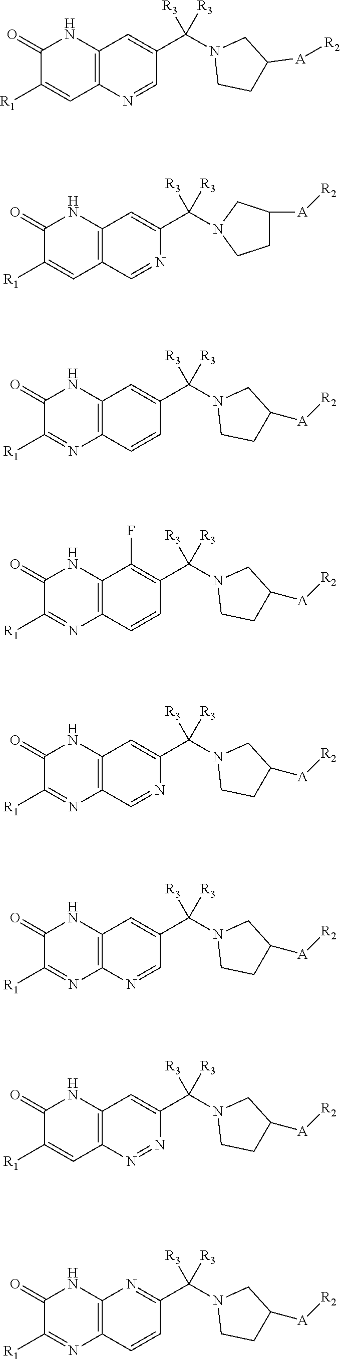 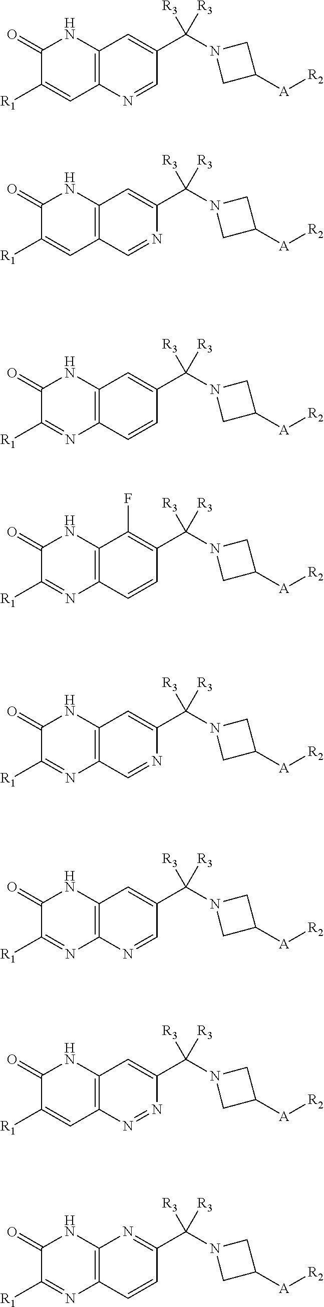
or a pharmaceutically acceptable salt thereof.

3. The compound according to claim 1 or 2, wherein,
A is selected from —O—,
B is selected from the group consisting of one substituted or unsubstituted 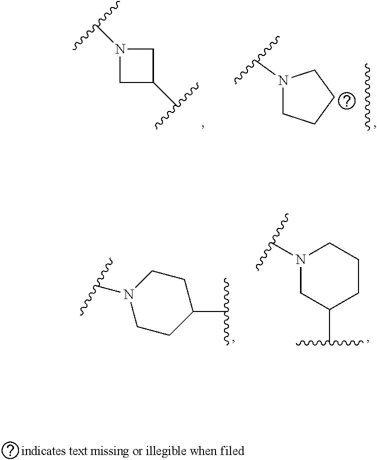
and the substituted group at any position of ring B is R₅;
R₁ is selected from the group consisting of C₁-C₃ alkyl, C₁-C₃ haloalkyl, C₁-C₃ alkoxy, C₃-C₅ cycloalkyl;
R₂ is 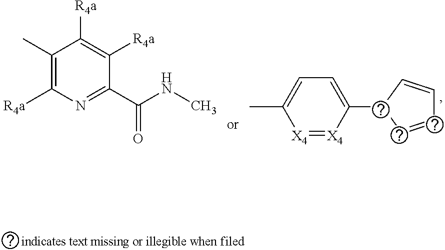
wherein one X₄ is N and one X₄ is CH;
each R₃ is independently selected from the group consisting of H or unsubstituted or substituted —C₁-C₆ alkyl, the substitutes of —C₁-C₆ alkyl is selected from the group consisting of H, —O—CH₃, —CN, —OH, or two R₃ are attached to form a C₃-C₆ cycloalkyl;
R₄ₐ is independently selected from the group consisting of H, CN, halogen, C₁-C₃ alkyl, —O—C₁-C₃ alkyl, C₁-C₃ haloalkyl, or —C₁-C₃ alkoxy; preferably, each R₄ₐ is independently selected from the group consisting of H, —CH₃, —CN, F;
R₅ is selected from the group consisting of —CH₃, —CH₂OH, or —F.

4. The compound according to claim 3, wherein the formula I is 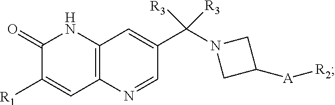
A is selected from —O—;
R₁ is selected from the group consisting of C₁-C₃ alkyl, C₃-C₅ cycloalkyl;
R₂ is 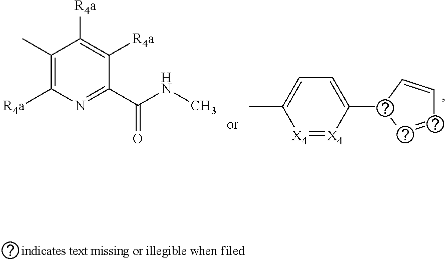
wherein one X₄ is N and one X₄ is CH;
each R₃ is independently selected from the group consisting of H or unsubstituted or substituted —C₁-C₆ alkyl, the substitutes of —C₁-C₆ alkyl is selected from the group consisting of H, —O—CH₃, —CN, —OH, or two R₃ are attached to form a C₃-C₅ cycloalkyl;
each R₄ₐ is independently selected from the group consisting of H, —CH₃, —CN, F.

5. The compound according to claim 4, wherein two R₃ are not H simultaneously.

6. The compound according to anyone claim of 5 or 6, wherein
R₂ is 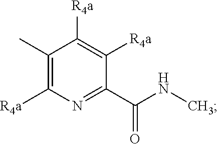
each R₄ₐ is independently selected from the group consisting of H, —CH₃, —CN, F;
each R₃ is independently selected from the group consisting of H or —C₁-C₃ alkyl, wherein two R₃ are not H simultaneously.

7. The compound according to claim 3, wherein the formula I is 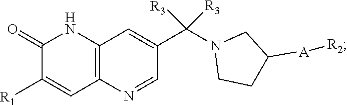
A is selected from —O—;
R₁ is selected from the group consisting of C₁-C₃ alkyl, C₃-C₅ cycloalkyl;
R₂ is 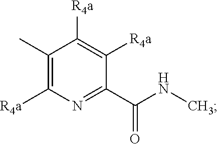
each R₃ is independently selected from the group consisting of H or unsubstituted or substituted —C₁-C₆ alkyl, the substitutes of —C₁-C₆ alkyl is selected from the group consisting of H, —O—CH₃, —CN, —OH, or two R₃ are attached to form a C₃-C₅ cycloalkyl;
each R₄ₐ is independently selected from the group consisting of H, —CH₃, —CN, F.

8. The compound according to claim 7, wherein,
R₁ is selected from the group consisting of C₁-C₃ alkyl;
each R₃ is independently selected from the group consisting of H or unsubstituted —C₁-C₃ alkyl;
each R₄ₐ is independently selected from the group consisting of H, —CH₃, F.

9. The compound according to anyone claim of 1 to 8, wherein the compound is selected from the group consisting of 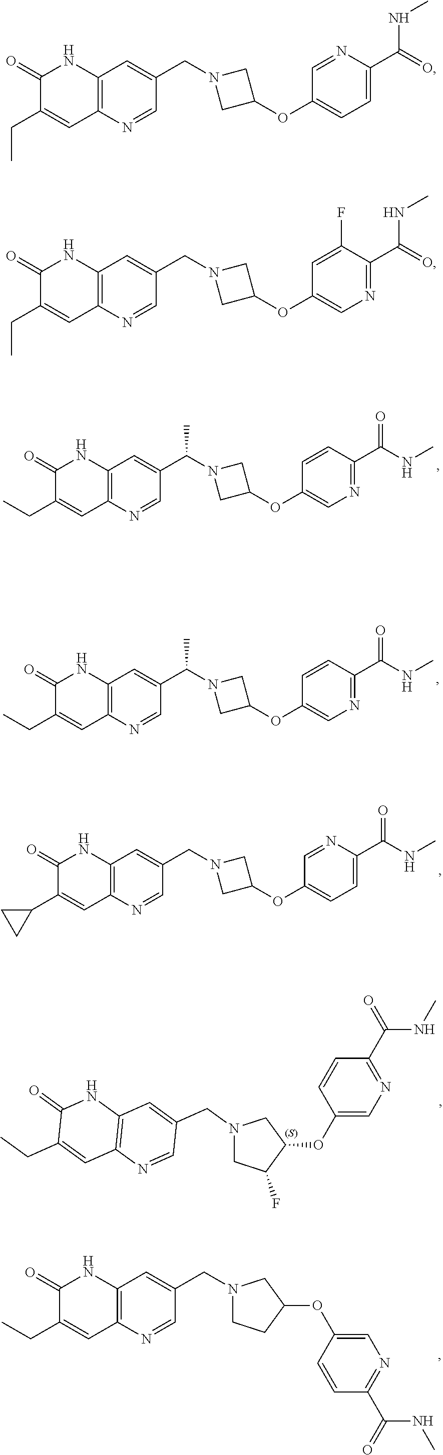 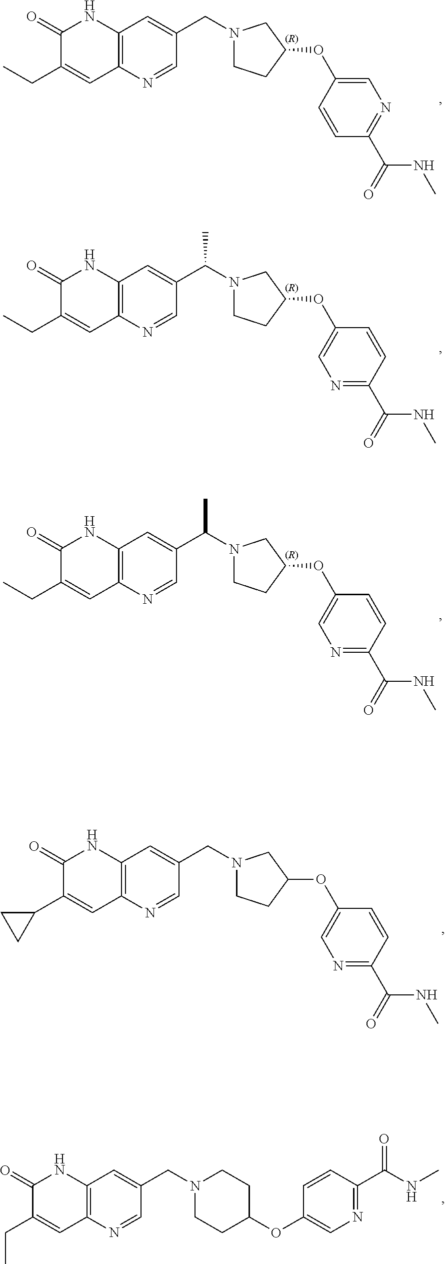 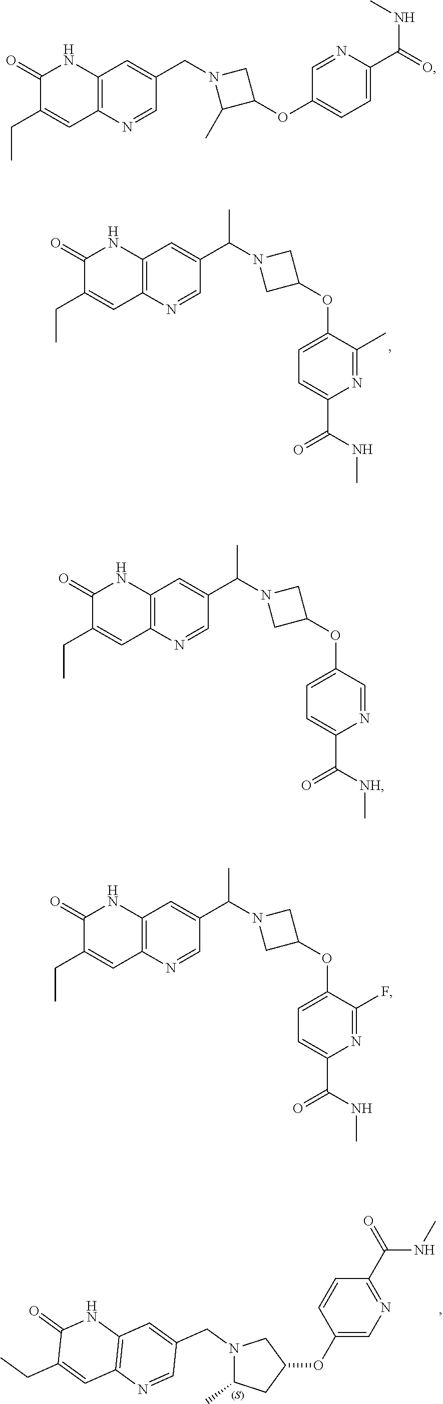 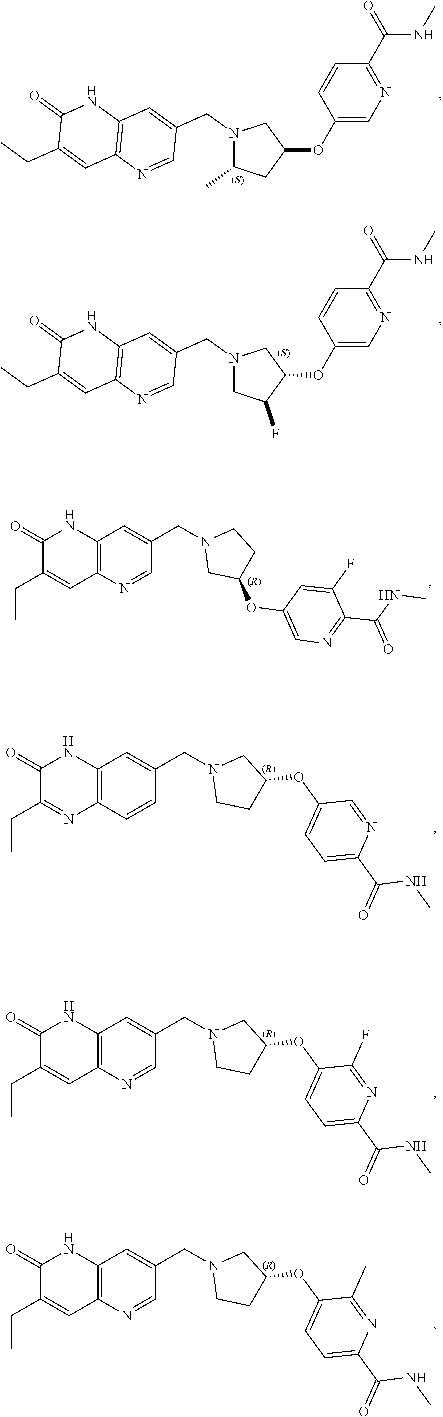 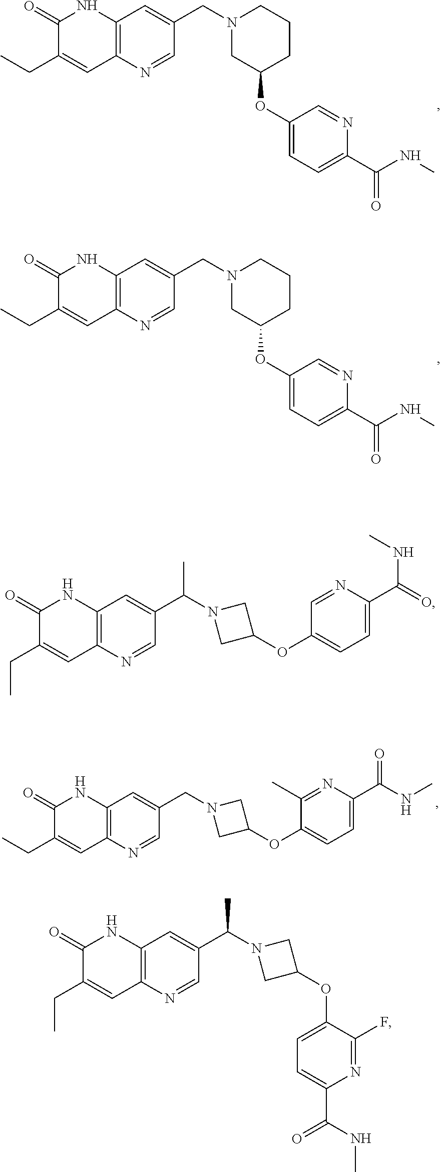 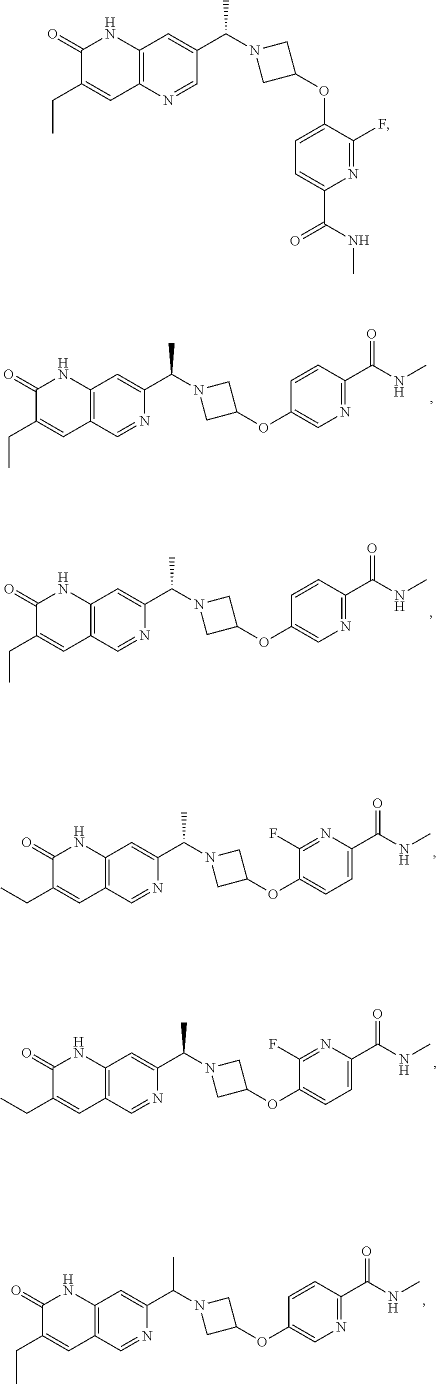 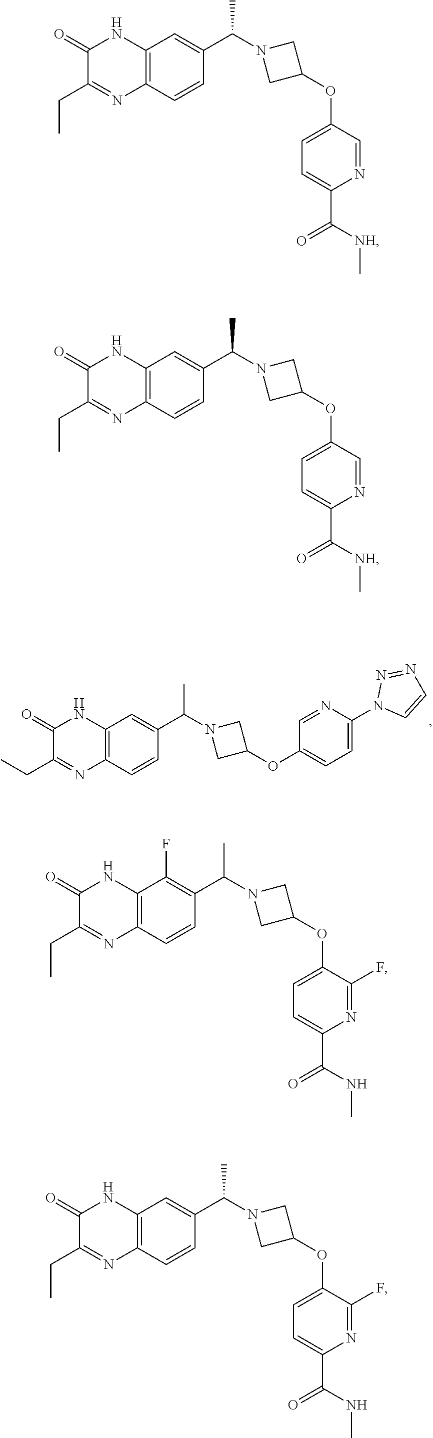 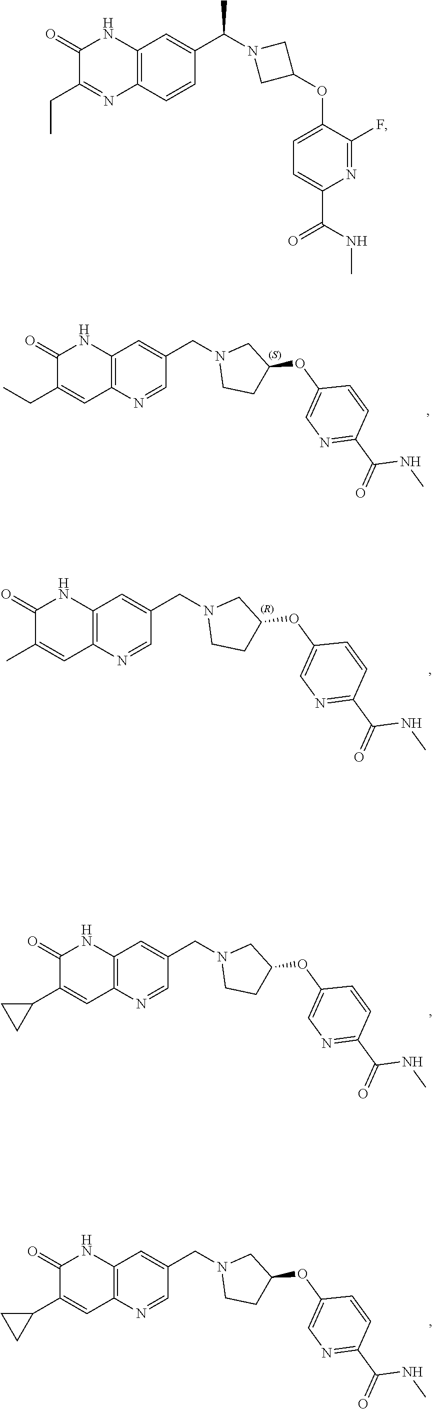 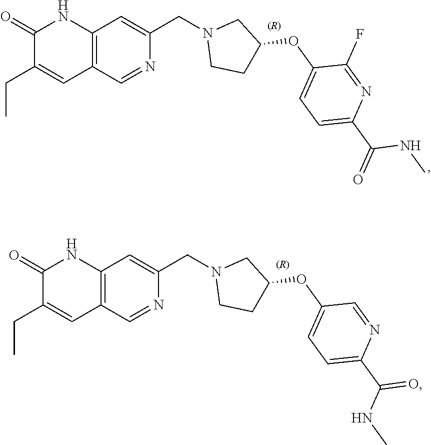
or a pharmaceutically acceptable salt thereof.

10. The compound according to anyone claim of 1 to 8, wherein the compound is selected from the group consisting of 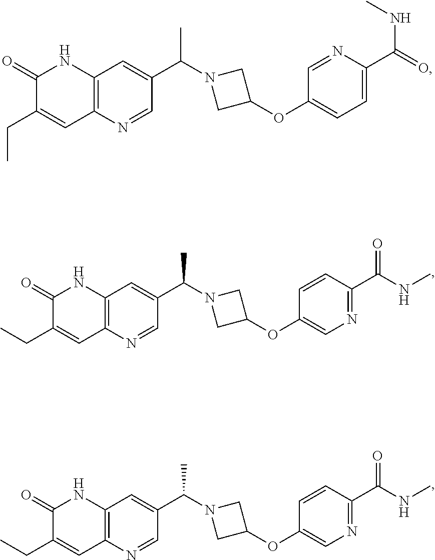
or a pharmaceutically acceptable salt thereof.

11. A pharmaceutical composition comprising a compound or a pharmaceutically acceptable salt thereof according to anyone of claims 1 to 10, and at least one pharmaceutically acceptable diluent, excipient or inert carrier.

12. A compound or a pharmaceutically acceptable salt thereof according to anyone of claims 1 to 10, for use as a medicament; or a method of treatment comprising administration of a therapeutically effective amount of a compound or a pharmaceutically acceptable salt thereof according to anyone of claims 1 to 10, to a patient in need thereof; or a use of a compound according to anyone of claims 1 to 10 in the manufacture of a medicament for use in the treatment of cancer.

13. The compound or the method or the use according to claim 12, wherein the patient in need has cancer.

14. The compound or the method or the use according to claim 13, wherein said cancer is deficient in HR dependent DNA DSB repair pathway.

15. The compound or the method or the use according to claim 14, wherein said cancer comprises one or more cancer cells having a reduced or abrogated ability to repair DNA DSB by HR relative to normal cells.

16. The compound or the method or the use according to claim 14 or 15, wherein said cancer cells have a BRCA1 or BRCA2 deficient phenotype.

17. The compound or the method or the use according to claim 16, wherein said cancer cells are deficient in BRCA1 or BRCA2.

18. The compound or the method or the use according to anyone of claims 16 to 17, wherein said individual is heterozygous for a mutation in a gene encoding a component of the HR dependent DNA DSB repair pathway.

19. The compound or the method or the use according to claim 18, wherein said individual is heterozygous for a mutation in BRCA1 and/or BRCA2.

20. The compound or the method or the use according to anyone of claims 12 to 19 wherein the cancer is selected from anyone of breast, ovary, pancreas, prostate, hematological, gastrointestinal, and lung cancer.

21. The compound or the method or the use according to anyone of claims 12 to 20, wherein the inhibition of PARP1 is beneficial in the treatment.
